(19) United States
(12) Patent Application Publication
(10) Pub. No.: US 2026/0042743 A1
(43) Pub. Date: Feb. 12, 2026

(54) SUBSTITUTED BENZIMIDAZOLES FOR TREATING VIRAL DISEASES

(71) Applicant: COUNCIL OF SCIENTIFIC AND INDUSTRIAL RESEARCH, New Delhi (IN)

(72) Inventors: Vinod Dinkar CHAUDHARI, Chandigarh (IN); Krishan Gopal THAKUR, Chandigarh (IN); Rajesh Prakash RINGE, Chandigarh (IN); Deshkanwar SINGH, Chandigarh (IN); Simran KAUR, Chandigarh (IN); Ravneet Singh CHAWLA, Chandigarh (IN); Akshay JOSHI, Chandigarh (IN)

(21) Appl. No.: 19/100,374

(22) PCT Filed: Aug. 1, 2023
(86) PCT No.: PCT/IN2023/050737

§ 371 Date: Jan. 31, 2025

(30) Foreign Application Priority Data
Aug. 1, 2022 (IN) 202211044091

(51) Int. Cl. C07D 401/04; A61K 31/4184; A61K 31/4439; A61K 31/454; A61K 45/06; A61P 31/14; C07D 235/30; C07D 401/12; C07D 405/12; C07D 409/12

(52) U.S. Cl. CPC C07D 401/04; A61K 31/4184; A61K 31/4439; A61K 31/454; A61K 45/06; A61P 31/14; C07D 235/30; C07D 401/12; C07D 405/12; C07D 409/12

(57) ABSTRACT

The invention relates to benzimidazole compounds, pharmaceutically acceptable salts thereof and its pharmaceutical compositions for reducing/treating viral infections. The present invention also relates to synthesis of such benzimidazole compounds. The present invention further relates to compositions which comprise such benzimidazole compounds or a pharmaceutically acceptable salt thereof, and a pharmaceutically acceptable carrier, optionally in combination. The compounds of the invention are useful in reducing/treating viral infections particularly coronavirus infections caused by SARS-CoV, MERS-CoV and SARS-CoV-2.

### FIELD OF THE INVENTION

The invention relates to benzimidazole compounds, pharmaceutically acceptable salts thereof and its pharmaceutical compositions. The present invention also relates to the synthesis of such benzimidazole compounds. The present invention further relates to compositions which comprise such benzimidazole compounds or a pharmaceutically acceptable salt thereof, and a pharmaceutically acceptable carrier, optionally in combination. The compounds of the invention are useful in reducing/treating viral infections particularly coronavirus infections caused by SARS-CoV, MERS-CoV, and SARS-CoV-2.

### BACKGROUND OF THE INVENTION

Over the last two decades, a number of viral epidemics have raised a serious issue in global public health risk. It includes Severe Acute Respiratory Syndrome Coronavirus (SARS-CoV) in 2002-2003, the Middle East Respiratory Syndrome Coronavirus (MERS-CoV) in 2012, and Ebola in 2014-2016. In 2019, a new viral disease emerged in Wuhan, China. The World Health Organization declared the outbreak to be a Public Health Emergency of International Concern on Jan. 30, 2020. The coronavirus causing the disease, as it was similar to SARS-CoV, was eventually named by the International Committee on Taxonomy of Viruses (ICTV) as COVID-19 (SARS-CoV-2). It was recognized as a pandemic on Mar. 11, 2020, due to its alarming level of spread and severity.

SARS-CoV-2 is a pathogenic RNA virus with an unusually large RNA genome, a nucleocapsid, and club-like spikes that project from their surface called spike (S) protein and are responsible for causing an ongoing COVID-19 pandemic. It belongs to the beta-coronavirus category which includes SARS-CoV and MERS-CoV. As of Mar. 11, 2022, more than 453,765,038 cases of COVID-19 have been reported worldwide, resulting in more than six million deaths and still counting. Several mutant variants of SARS-CoV-2 including delta and recently found Omicron, deltacron, having higher infectivity or disease severity, have been reported. To date, there is no specific therapeutic drug or targeted therapeutics to treat or prevent COVID-19. The demand for new, effective, and safe antiviral strategies has become an important problem to solve in recent years due to the rising prevalence of viral infections such as HIV and hepatitis B and C and the emergence of new viruses or viral strains such as the SARS coronavirus.

Currently available antiviral drugs, such as the nucleoside reverse transcriptase inhibitors (NRTIs) used to inhibit viral replication of specific pathogenic viruses, have resulted in recognizable improvements in the ability to control infections with these pathogens and to improve the quality and length of life of infected individuals. Vaccines, although successful, may not be effective in countering mutant strains. Vaccines are helpful as prophylaxis, and still, there is an unmet medical need for an effective treatment of COVID-19. The most promising therapies including experimental antiviral compounds called Remdesivir and Favipiravir; the malaria medications chloroquine and hydroxychloroquine; a combination of two HIV/AIDS drugs, Darunavir/cobicistat and lopinavir/ritonavir; and that same combination plus interferon-beta, an immune system messenger and plasma therapy did not yield promising results in the clinical trials. Few potent monoclonal antibodies have been approved by FDA for the treatment of COVID-19; however, the therapy fails against Omicron and may likely—happen for future novel variants. Despite multiple investigational drug approval for COVID-19 or SARS-CoV-2 infections, the treatment at all points in the course of infection remains an unmet clinical need. Currently, available classes of antiviral agents have limited utilities due to their narrow scope of activities against different viruses and/or problems with significant drug-induced toxicities. In addition, the modes of action of the NRTIs and other drugs in current clinical use predispose to the development of drug resistance through viral mutations that prevent their widespread use to achieve treatment or prevention goals.

The design and discovery of new antiviral drugs can be directed against the targets as described above. Drugs that inhibit viral proteins are more likely to be virus specific and are more prone to the development of resistance. Thus, there is an urgent need for broad-spectrum antiviral drugs that can be used to treat numerous viral diseases. The demand for new effective and safe antiviral strategies has become an important problem to solve in recent years due to the rising prevalence of viral infections such as coronavirus, HIV, Ebola, Nipah, H1N1, hepatitis B/C, etc.

### SUMMARY OF THE INVENTION

The present invention provides a compound of general formula (I), a stereoisomer thereof, or a pharmaceutically acceptable salt thereof,

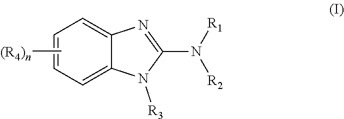

wherein,
R₁ and R₂, are independently selected from hydrogen, substituted or unsubstituted alkyl, substituted or unsubstituted cycloalkyl, substituted or unsubstituted aryl, substituted or unsubstituted heteroaryl, substituted or unsubstituted heterocyclic ring, wherein one or more substituents are selected from halogen, alkyl, alkoxy, oxo, nitro, trifluoromethyl, trifluoromethoxy, aryl and heteroaryl; or R₁ and R₂ together with the nitrogen to which they are attached, may form a substituted or unsubstituted 3 to 7 membered saturated or unsaturated heterocyclic ring, wherein the substituents are selected from halogen, alkyl, alkoxy, oxo, nitro, trifluoromethyl, trifluoromethoxy, aryl and heteroaryl; and R₁ and R₂ are not simultaneously hydrogen;
R₃ is selected from the group consisting of hydrogen, substituted or unsubstituted alkyl, substituted or unsubstituted cycloalkyl; alkoxy, carboxy, carboxy ester, substituted or unsubstituted aryl, and substituted or unsubstituted heteroaryl;
R₄ which may be same or different at each occurrence, is independently selected from halogen, cyano, nitro, hydroxyl, trifluoromethyl, trifluoromethoxy, substituted or unsubstituted alkyl, substituted haloalkyl, substituted or unsubstituted cycloalkyl, NRₐR_{b}, —C(O)NRₐR_{b}, —NRₐC(O)R_{b}, —NRₐS(O)₀₋₂R_{b}, —S(O)₀₋₂Rₐ, and —S(O)₀₋₂NRₐR_{b}; Rₐ and R_{b}, which may be same or different at each occurrence, are independently selected from hydrogen, substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, and substituted or unsubstituted heterocyclylalkyl; or Rₐ and R_{b} together with the nitrogen atom to which they are attached, may form a substituted or unsubstituted, saturated or unsaturated 3 to 10 membered cyclic ring, and
‘n’ is an integer ranging from 1 to 4.

In a preferred embodiment of the present invention, the said compound is selected from the group consisting of:
N-(3,5-bis(trifluoromethyl)phenyl)-4,6-bis(trifluoromethyl)-1H-benzo[d]imidazol-2-amine;
N-(4-chlorophenyl)-4,6-bis(trifluoromethyl)-1H-benzo[d]imidazol-2-amine;
N-phenyl-4,6-bis(trifluoromethyl)-1H-benzo[d]imidazol-2-amine;
N-(3-bromo-4-methoxyphenyl)-4,6-bis(trifluoromethyl)-1H-benzo[d]imidazol-2-amine;
N-(pyridin-3-yl)-4,6-bis(trifluoromethyl)-1H-benzo[d]imidazol-2-amine;
N-benzyl-4,6-bis(trifluoromethyl)-1H-benzo[d]imidazole-2-amine;
N-(tetrahydro-2H-pyran-4-yl)-4,6-bis(trifluoromethyl)-1H-benzo[d]imidazol-2-amine;
5,6-difluoro-N-phenyl-1H-benzo[d]imidazol-2-amine;
N-(3-bromo-4-methoxyphenyl)-5,6-difluoro-1H-benzo[d]imidazol-2-amine; 5,6-difluoro-N-(3-(trifluoromethyl)phenyl)-1H-benzo[d]imidazol-2-amine;
N-(3,5-bis(trifluoromethyl)phenyl)-5,6-difluoro-1H-benzo[d]imidazol-2-amine; 5,6-difluoro-N-(4-(trifluoromethyl)phenyl)-1H-benzo[d]imidazol-2-amine;
N-benzyl-5,6-difluoro-1H-benzo[d]imidazol-2-amine;
4,6-bis(trifluoromethyl)-N-(3-(trifluoromethyl)phenyl)-1H-benzo[d]imidazol-2-amine;
N-isopropyl-4,6-bis(trifluoromethyl)-1H-benzo[d]imidazol-2-amine;
2-(piperidin-1-yl)-4,6-bis(trifluoromethyl)-1H-benzo[d]imidazole;
N-(naphthalen-2-yl)-5,7-bis(trifluoromethyl)-1H-benzo[d]imidazol-2-amine;
N-(2-isopropylphenyl)-4,6-bis(trifluoromethyl)-1H-benzo[d]imidazol-2-amine;
(R)—N-(1-(naphthalen-1-yl)ethyl)-4,6-bis(trifluoromethyl)-1H-benzo[d]imidazol-2-amine;
3-((4,6-bis(trifluoromethyl)-1H-benzo[d]imidazol-2-yl)amino)tetrahydrothiophene 1,1-dioxide;
N-(1-methylcyclobutyl)-4,6-bis(trifluoromethyl)-1H-benzo[d]imidazol-2-amine;
N-(3-(trifluoromethoxy)phenyl)-4,6-bis(trifluoromethyl)-1H-benzo[d]imidazol-2-amine;
N-(2-fluorophenyl)-4,6-bis(trifluoromethyl)-1H-benzo[d]imidazol-2-amine;
N-(3-fluorophenyl)-4,6-bis(trifluoromethyl)-1H-benzo[d]imidazole-2-amine;
N-(4-fluorophenyl)-4,6-bis(trifluoromethyl)-1H-benzo[d]imidazol-2-amine;
N-(3,5-difluorophenyl)-4,6-bis(trifluoromethyl)-1H-benzo[d]imidazol-2-amine;
4,6-bis(trifluoromethyl)-N-(4-(trifluoromethyl)phenyl)-1H-benzo[d]imidazol-2-amine;
4,6-bis(trifluoromethyl)-N-(2-(trifluoromethyl)phenyl)-1H-benzo[d]imidazol-2-amine;
N-(3,5-bis(trifluoromethyl)phenyl)-6-(trifluoromethoxy)-1H-benzo[d]imidazol-2-amine;
6-(trifluoromethoxy)-N-(3-(trifluoromethyl)phenyl)-1H-benzo[d]imidazol-2-amine;
N-(3,5-bis(trifluoromethyl)phenyl)-6-(trifluoromethyl)-1H-benzo[d]imidazol-2-amine;
6-(trifluoromethyl)-N-(3-(trifluoromethyl)phenyl)-1H-benzo[d]imidazol-2-amine;
6-(trifluoromethyl)-N-(4-(trifluoromethyl)phenyl)-1H-benzo[d]imidazol-2-amine;
N-(3,5-bis(trifluoromethyl)phenyl)-4-(trifluoromethyl)-1H-benzo[d]imidazol-2-amine;
4-(trifluoromethyl)-N-(3-(trifluoromethyl)phenyl)-1H-benzo[d]imidazol-2-amine;
4-(trifluoromethyl)-N-(4-(trifluoromethyl)phenyl)-1H-benzo[d]imidazole-2-amine;
N-(3,5-bis(trifluoromethyl)phenyl)-6-methoxy-1H-benzo[d]imidazol-2-amine;
5,6-difluoro-1-methyl-N-phenyl-1H-benzo[d]imidazole-2-amine;
N-(3,5-bis(trifluoromethyl)phenyl)-5,6-difluoro-1-methyl-1H-benzo[d]imidazol-2-amine;
N-methyl-4,6-bis(trifluoromethyl)-N-(3-(trifluoromethyl)phenyl)-1H-benzo[d]imidazol-2-amine;
N-(3,5-bis(trifluoromethyl)phenyl)-N-methyl-4,6-bis(trifluoromethyl)-1H-benzo[d]imidazol-2-amine;
N-methyl-7-(trifluoromethyl)-N-(3-(trifluoromethyl)phenyl)-1H-benzo[d]imidazol-2-amine;
N-methyl-7-(trifluoromethyl)-N-(4-(trifluoromethyl)phenyl)-1H-benzo[d]imidazol-2-amine; and
N-(3,5-bis(trifluoromethyl)phenyl)-N-methyl-7-(trifluoromethyl)-11H-benzo[d]imidazole-2-amine.

The present invention provides a process for preparing the compound of general formula (I)

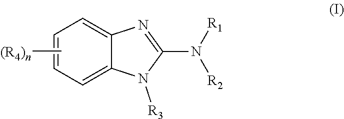

wherein, R₁ and R₂, are independently selected from hydrogen, substituted or unsubstituted alkyl, substituted or unsubstituted cycloalkyl, substituted or unsubstituted aryl, substituted or unsubstituted heteroaryl, substituted or unsubstituted heterocyclic ring, wherein one or more substituents are selected from halogen, alkyl, alkoxy, oxo, nitro, trifluoromethyl, trifluoromethoxy, aryl and heteroaryl; or R₁ and R₂ together with the nitrogen to which they are attached, may form a substituted or unsubstituted 3 to 7 membered saturated or unsaturated heterocyclic ring, wherein the substituents are selected from the group of halogen, alkyl, alkoxy, oxo, nitro, trifluoromethyl, trifluoromethoxy, aryl and heteroaryl; and R₁ and R₂ are not simultaneously hydrogen;
R₃ is selected from the group consisting of hydrogen, substituted or unsubstituted alkyl, substituted or unsubstituted cycloalkyl; alkoxy, carboxy, carboxy ester, substituted or unsubstituted aryl and substituted or unsubstituted heteroaryl;
R₄ which may be same or different at each occurrence, is independently selected from halogen, cyano, nitro, hydroxyl, trifluoromethyl, trifluoromethoxy, substituted or unsubstituted alkyl, substituted or unsubstituted haloalkyl, substituted or unsubstituted cycloalkyl, NRₐR_{b}, —C(O)NRₐR_{b}, —NRₐC(O)R_{b}, —NRₐS(O)₀₋₂R_{b}, —S(O)₀₋₂Rₐ, and—S(O)₀₋₂NRₐR_{b};
Rₐ and R_{b}, which may be same or different at each occurrence, are independently selected from hydrogen, substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, and substituted or unsubstituted heterocyclylalkyl; or Rₐ and R_{b} together with the nitrogen atom to which they are attached, may form a substituted or unsubstituted, saturated or unsaturated 3 to 10 membered cyclic ring, and
‘n’ is an integer ranging from 1 to 4, comprising the steps of:
a. acetylation of substituted aniline for formula (1) in the presence of an anhydride to produce a compound of formula (2);

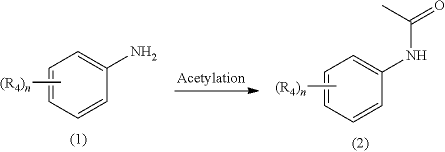

b. nitration of the compound of formula (2) to produce an acetamide compound of formula (3);

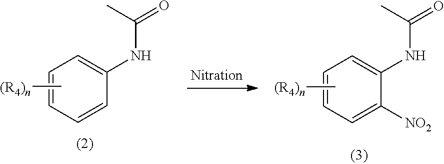

c. treating the acetamide compound of formula (3) with a base to produce a compound of formula (4);

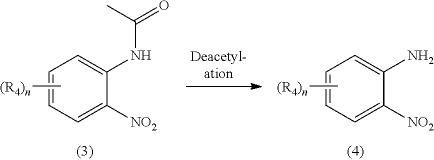

d. reducing the nitro group of the compound of formula (4) by a reducing agent to produce a diamine of formula (5);

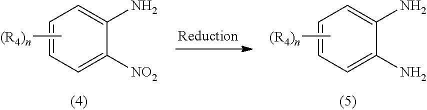

e. cyclization of the diamine of formula (5) with a coupling agent in the presence or absence of catalytic amount of a base to produce a compound of formula (6);

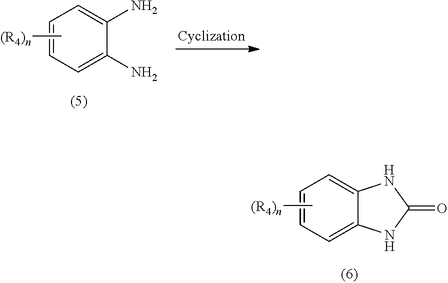

f. halogenation of the compound of formula (6) to produce a compound of formula (7);

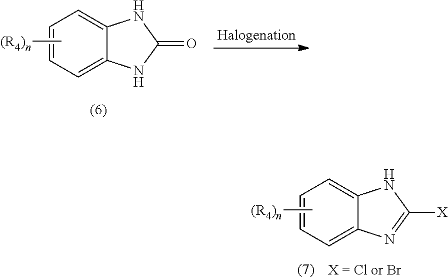

g. optional N-substitution of the compound of formula (7) using alkylating/arylating agent in basic condition to obtain a compound of formula (9);

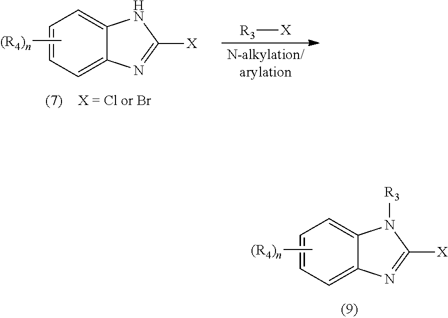

h. nucleophilic substitution of compound of formula (7) or (9) with a substituted or unsubstituted aniline or amine (8) in acidic conditions to obtain a compound of Formula (I).

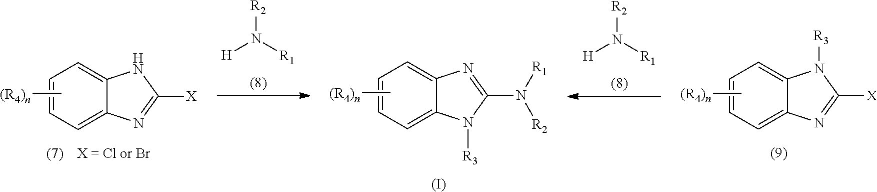

In a preferred embodiment of the present invention the anhydride is acetic anhydride.

In a preferred embodiment of the present invention, the base used in step c is sodium hydroxide or potassium hydroxide.

In a preferred embodiment of the present invention, the reducing agent is selected from H₂, Pd/C, Iron in acetic acid and SnCl₂.

In a preferred embodiment of the present invention, the coupling agent is carbonyldiimidazole or triphosgene.

In a preferred embodiment of the present invention, the halogenation is carried out using phosphoryl chloride or phosphoryl bromide.

The present invention provides a pharmaceutical composition comprising the compound of general Formula (I) as claimed in claim 1 and one or more pharmaceutically acceptable excipients for treating coronavirus infections caused by SARS-CoV, MERS-CoV, SARS-CoV-2, and/or other viral infections.

In a preferred embodiment of the present invention the pharmaceutical composition which further comprises an effective amount of an antiviral compound.

In another embodiment of the present invention pharmaceutical composition comprising the compound of general Formula (I) along with one or more antiviral compound are prepared.

These and other features, aspects, and advantages of the present subject matter will be better understood with reference to the following description and appended claims. This summary is provided to introduce a selection of concepts in a simplified form. This summary is not intended to identify key features or essential features of the claimed subject matter, nor is it intended to be used to limit the scope of the claimed subject matter.

### DETAILED DESCRIPTION OF THE INVENTION

The invention will now be described in detail in connection with certain preferred and optional embodiments, so that various aspects thereof may be more fully understood and appreciated.

For convenience, before further description of the present disclosure, certain terms employed in the specification, and examples are delineated here. These definitions should be read in the light of the remainder of the disclosure and understood as by a person of skill in the art. The terms used herein have the meanings recognized and known to those of skill in the art, however, for convenience and completeness, particular terms and their meanings are set forth below.

The articles “a”, “an” and “the” are used to refer to one or to more than one (i.e., to at least one) of the grammatical object of the article.

The terms “comprise” and “comprising” are used in the inclusive, open sense, meaning that additional elements may be included. It is not intended to be construed as “consists of only”.

Throughout this specification, unless the context requires otherwise the word “comprise”, and variations such as “comprises” and “comprising”, will be understood to imply the inclusion of a stated element or step or group of element or steps but not the exclusion of any other element or step or group of element or steps.

Ratios, concentrations, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited.

### Definitions and Abbreviations

Unless otherwise stated, the following terms used in the specification and claims have the meanings given below.

For purposes of interpreting the specification, the following definitions will apply and whenever appropriate, terms used in the singular will also include the plural and vice versa.

The terms “halogen” or “halo” means fluorine, chlorine, bromine, or iodine. The term “alkyl” refers to an alkane derived hydrocarbon radical that includes solely carbon and hydrogen atoms in the backbone, contains no unsaturation, has from one to six carbon atoms, and is attached to the remainder of the molecule by a single bond, for example C₁₋₆ alkyl, representative groups include e.g., methyl, ethyl, n-propyl, 1-methylethyl (isopropyl), n-butyl, n-pentyl and the like. Unless set forth or recited to the contrary, all alkyl groups described or claimed herein may be straight chain or branched.

The term “alkenyl” refers to a hydrocarbon radical containing from 2 to 10 carbon atoms and including at least one carbon-carbon double bond. Non-limiting Examples of alkenyl groups include, for example C₂₋₆ alkenyl, C₂₋₄ alkenyl, ethenyl, 1-propenyl, 2-propenyl (allyl), and the like. Unless set forth or recited to the contrary, all alkenyl groups described or claimed herein may be straight chain or branched.

The term “alkynyl” refers to a hydrocarbon radical containing 2 to 10 carbon atoms and including at least one carbon-carbon triple bond. Non-limiting Examples of alkynyl groups include, for example C₂₋₆ alkynyl, C₂₋₄ alkynyl, ethynyl, propynyl, butynyl and the like. Unless set forth or recited to the contrary, all alkynyl groups described or claimed herein may be straight chain or branched.

The term “haloalkyl” refers to an alkyl group as defined above that is substituted by one or more halogen atoms as defined above. For example, C₁₋₆ haloalkyl or C₁₋₄ haloalkyl. Suitably, the haloalkyl may be monohaloalkyl, dihaloalkyl or polyhaloalkyl including perhaloalkyl. A monohaloalkyl can have one iodine, bromine, chlorine or fluorine atom. Dihaloalkyl and polyhaloalkyl groups can be substituted with two or more of the same halogen atoms or a combination of different halogen atoms. Suitably, a polyhaloalkyl is substituted with up to 12 halogen atoms. Non-limiting examples of a haloalkyl include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl, dichloropropyl and the like. A perhaloalkyl refers to an alkyl having all hydrogen atoms replaced with halogen atoms. Unless set forth or recited to the contrary, all haloalkyl groups described or claimed herein may be straight chain or branched.

The term “alkoxy” denotes an alkyl group attached via an oxygen linkage to the rest of the molecule. Representative examples of such groups are —OCH₃ and —OC₂H₅. Unless set forth or recited to the contrary, all alkoxy groups described or claimed herein may be straight chain or branched.

The term “alkoxyalkyl” refers to an alkoxy group as defined above directly bonded to an alkyl group as defined above, e.g., —CH₂—O—CH₃, —CH₂—O—CH₂CH₃, —CH₂CH₂—O—CH₃ and the like.

The term “cycloalkyl” refers to a non-aromatic mono or multicyclic ring system having 3 to 12 carbon atoms, such as C₃₋₁₀ cycloalkyl, C₃₋₆ cycloalkyl, cyclopropyl, cyclobutyl, cyclopentyl, cyclohexyl and the like. Examples of multicyclic cycloalkyl groups include, but are not limited to, perhydronaphthyl, adamantyl and norbornyl groups, bridged cyclic groups or spirobicyclic groups, e.g., spiro(4,4)non-2-yl and the like.

The term “aryl” refers to an aromatic radical having 6 to 14 carbon atoms, including monocyclic, bicyclic, and tricyclic aromatic systems, such as phenyl, naphthyl, tetrahydronaphthyl, indanyl, biphenyl and the like.

The term “heterocyclic ring” or “heterocyclyl ring” or “heterocyclyl”, unless otherwise specified, refers to substituted or unsubstituted non-aromatic 3 to 15 membered ring which consists of carbon atoms and with one or more heteroatom(s) independently selected from N, O or S. The heterocyclic ring may be a mono, bi or tricyclic ring system, which may include fused, bridged or spiro ring systems and the nitrogen, carbon, oxygen or sulfur atoms in the heterocyclic ring may be optionally oxidized to various oxidation states. In addition, the nitrogen atom may be optionally quaternized, the heterocyclic ring or heterocyclyl may optionally contain one or more olefinic bond, and one or two carbon atoms/in the heterocyclic ring or heterocyclyl may be interrupted with —CF₂—, —C(O)—, —S(O)—, —S(O)₂ etc. In addition, heterocyclic ring may also be fused with aromatic ring. Non-limiting Examples of heterocyclic rings include azetidinyl, benzopyranyl, chromanyl, decahydroisoquinolyl, indolinyl, isoindolinyl, isochromanyl, isothiazolidinyl, isoxazolidinyl, morpholinyl, oxazolinyl, oxazolidinyl, 2-oxopiperazinyl, 2-oxopiperidinyl, 2-oxopyrrolidinyl, 2-oxoazepinyl, octahydroindolyl, octahydroisoindolyl, perhydroazepinyl, piperazinyl, 4-piperidonyl, pyrrolidinyl, piperidinyl, phenothiazinyl, phenoxazinyl, quinuclidinyl, tetrahydroisquinolyl, tetrahydrofuryl, tetrahydropyranyl, thiazolinyl, thiazolidinyl, thiamorpholinyl, thiamorpholinylsulfoxide, thiamorpholinylsulfoneindoline, benzodioxole, tetrahydroquinoline, tetrahydrobenzopyran and the like. The heterocyclic ring may be attached by any atom of the heterocyclic ring that results in the creation of a stable structure.

The term “heteroaryl” unless otherwise specified, refers to a substituted or unsubstituted 5 to 14 membered aromatic heterocyclic ring with one or more heteroatom/s independently selected from N, O or S. The heteroaryl may be a mono, bi or tricyclic ring system. The heteroaryl ring may be attached by any atom of the heteroaryl ring that results in the creation of a stable structure. Non-limiting Examples of a heteroaryl ring include oxazolyl, isoxazolyl, imidazolyl, furyl, indolyl, isoindolyl, pyrrolyl, triazolyl, triazinyl, tetrazolyl, thienyl, thiazolyl, isothiazolyl, pyridyl, pyrimidinyl, pyrazinyl, pyridazinyl, benzofuranyl, benzothiazolyl, benzoxazolyl, benzimidazolyl, benzothienyl, carbazolyl, quinolinyl, isoquinolinyl, quinazolinyl, cinnolinyl, naphthyridinyl, pteridinyl, purinyl, quinoxalinyl, quinolyl, isoquinolyl, thiadiazolyl, indolizinyl, acridinyl, phenazinyl, phthalazinyl and the like.

The term “treating” or “treatment” of a state, disorder, infections or condition includes: (a) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition; (b) inhibiting the state, disorder or condition, i.e., arresting or reducing the development of the disease or at least one clinical or subclinical symptom thereof; (c) lessening the disease, disorder or condition or at least one of its clinical or subclinical symptoms or (d) relieving the disease, i.e., causing regression of the state, disorder or condition or at least one of its clinical or subclinical symptoms.

As used herein, the term “effective amount” refers to the amount of each active agent required to confer the desired effect (e.g. Antiviral) on the subject, either alone or in combination with one or more other active agents. An effective amount varies, as recognized by those skilled in the art, depending on the particular condition being treated, the severity of the condition, the individual patient parameters including age, size, physical condition, weight and gender, the nature of concurrent therapy (if any), the duration of the treatment, the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgement. It will be understood by those of ordinary skill in the art, however, that a patient may insist upon a lower dose or tolerable dose for medical reasons, psychological reasons or for virtually any other reasons.

A “therapeutically effective amount” means the amount of a compound that, when administered to a subject for treating a disease, disorder, or condition, is sufficient to cause the effect in the subject, which is the purpose of the administration. The “therapeutically effective amount” will vary depending on the compound, the disease and its severity, and the age, weight, physical condition, and responsiveness of the subject to be treated.

In an embodiment of the present disclosure, there is provided a compound of general formula (I), a stereoisomer thereof, or a pharmaceutically acceptable salt thereof,

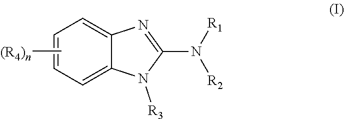

wherein,
R₁ and R₂, are independently selected from hydrogen, substituted or unsubstituted alkyl, substituted or unsubstituted cycloalkyl, substituted or unsubstituted aryl, substituted or unsubstituted heteroaryl, substituted or unsubstituted heterocyclic ring, wherein one or more substituents are selected from halogen, alkyl, alkoxy, oxo, nitro, trifluoromethyl, trifluoromethoxy, aryl and heteroaryl; or R₁ and R₂ together with the nitrogen to which they are attached, may form a substituted or unsubstituted 3 to 7 membered saturated or unsaturated heterocyclic ring, wherein the substituents are selected from halogen, alkyl, alkoxy, oxo, nitro, trifluoromethyl, trifluoromethoxy, aryl and heteroaryl; and R₁ and R₂ are not simultaneously hydrogen;
R₃ is selected from the group consisting of hydrogen, substituted or unsubstituted alkyl, substituted or unsubstituted cycloalkyl; alkoxy, carboxy, carboxy ester, substituted or unsubstituted aryl and substituted or unsubstituted heteroaryl;
R₄ which may be same or different at each occurrence, is independently selected from halogen, cyano, nitro, hydroxyl, trifluoromethyl, trifluoromethoxy, substituted or unsubstituted alkyl, substituted haloalkyl, substituted or unsubstituted cycloalkyl, NRₐR_{b}, —C(O)NRₐR_{b}, —NRₐC(O)R_{b}, —NRₐS(O)₀₋₂R_{b}, —S(O)₀₋₂Rₐ, and —S(O)₀₋₂NRₐR_{b}; Rₐ and R_{b}, which may be same or different at each occurrence, are independently selected from hydrogen, substituted or unsubstituted alkyl, substituted or unsubstituted cycloalkyl, substituted or unsubstituted cycloalkylalkyl, substituted or unsubstituted aryl, substituted or unsubstituted arylalkyl, substituted or unsubstituted heteroaryl, substituted or unsubstituted heteroarylalkyl, substituted or unsubstituted heterocyclyl, and substituted or unsubstituted heterocyclylalkyl; or Rₐ and R_{b} together with the nitrogen atom to which they are attached, may form a substituted or unsubstituted, saturated or unsaturated 3 to 10 membered cyclic ring, and
‘n’ is an integer ranging from 1 to 4.

In another embodiment of the present disclosure, there is provided a compound of general formula (I), a stereoisomer thereof, or a pharmaceutically acceptable salt thereof,

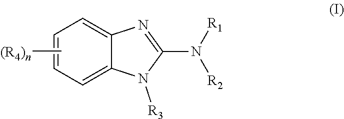

wherein,
R₁ and R₂, are independently selected from hydrogen, substituted or unsubstituted C₁₋₁₂ alkyl, substituted or unsubstituted C₃₋₁₂ cycloalkyl, substituted or unsubstituted C₆₋₁₆ aryl, substituted or unsubstituted C₅₋₁₆ heteroaryl, substituted or unsubstituted C₃₋₁₆ heterocyclic ring, wherein one or more substituents are selected from halogen, alkyl, alkoxy, oxo, nitro, trifluoromethyl, trifluoromethoxy, aryl and heteroaryl; or R₁ and R₂ together with the nitrogen to which they are attached, may form a substituted or unsubstituted 3 to 7 membered saturated or unsaturated heterocyclic ring, wherein the substituents are selected from halogen, C₁₋₁₂ alkyl, C₁₋₁₂ alkoxy, oxo, nitro, trifluoromethyl, trifluoromethoxy, C₆₋₁₆ aryl and C₅₋₁₆ heteroaryl; and R₁ and R₂ are not simultaneously hydrogen;
R₃ is selected from the group consisting of hydrogen, substituted or unsubstituted C₁₋₁₂ alkyl, substituted or unsubstituted C₁₋₁₂ cycloalkyl; C₁₋₁₂ alkoxy, carboxy, carboxy ester, substituted or unsubstituted C₆₋₁₆ aryl and substituted or unsubstituted C₅₋₁₆ heteroaryl;
R₄ which may be same or different at each occurrence, is independently selected from halogen, cyano, nitro, hydroxyl, trifluoromethyl, trifluoromethoxy, substituted or unsubstituted C₁₋₁₂ alkyl, substituted C₁₋₁₂ haloalkyl, substituted or unsubstituted C₃₋₁₂ cycloalkyl, NRₐR_{b}, —C(O)NRₐR_{b}, —NRₐC(O)R_{b}, —NRₐS(O)₀₋₂R_{b}, —S(O)₀₋₂Rₐ, and—S(O)₀₋₂NRₐR_{b}; Rₐ and R_{b}, which may be same or different at each occurrence, are independently selected from hydrogen, substituted or unsubstituted C₁₋₁₂ alkyl, substituted or unsubstituted C₃₋₁₂ cycloalkyl, substituted or unsubstituted C₃₋₁₂ cycloalkylalkyl, substituted or unsubstituted C₆₋₁₂ aryl, substituted or unsubstituted C₁₋₁₈ arylalkyl, substituted or unsubstituted C₅₋₁₆ heteroaryl, substituted or unsubstituted C₁₋₁₈ heteroarylalkyl, substituted or unsubstituted C₃₋₁₂ heterocyclyl, and substituted or unsubstituted C₁₋₁₈ heterocyclylalkyl; or Rₐ and R_{b} together with the nitrogen atom to which they are attached, may form a substituted or unsubstituted, saturated or unsaturated 3 to 10 membered cyclic ring, and
‘n’ is an integer ranging from 1 to 3.

In yet another embodiment of the present disclosure, there is provided a compound of general formula (I), a stereoisomer thereof, or a pharmaceutically acceptable salt thereof,

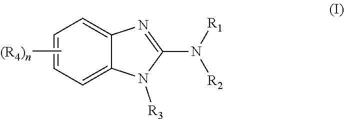

wherein,
R₁ and R₂, are independently selected from hydrogen, substituted or unsubstituted C₁₋₆ alkyl, substituted or unsubstituted C₃₋₇ cycloalkyl, substituted or unsubstituted C₆₋₁₀ aryl, substituted or unsubstituted C₅₋₇ heteroaryl, substituted or unsubstituted C₃₋₇ heterocyclic ring, wherein one or more substituents are selected from halogen, alkyl, alkoxy, oxo, nitro, trifluoromethyl, trifluoromethoxy, aryl and heteroaryl; or
R₁ and R₂ together with the nitrogen to which they are attached, may form a substituted or unsubstituted 4 to 6 membered saturated or unsaturated heterocyclic ring, wherein the substituents are selected from halogen, C₂₋₆ alkyl, C₂₋₆ alkoxy, oxo, nitro, trifluoromethyl, trifluoromethoxy, C₃₋₁₀ aryl and C₃₋₇ heteroaryl; and R₁ and R₂ are not simultaneously hydrogen;
R₃ is selected from the group consisting of hydrogen, substituted or unsubstituted C₁₋₆ alkyl, and C₁₋₆ alkoxy;
R₄ which may be same or different at each occurrence, is independently selected from the group consisting of halogen, trifluoromethyl, and trifluoromethoxy; and
‘n’ is an integer ranging from 1 to 3.

### Pharmaceutically Acceptable Salts

The compounds of the invention may form salts with acid or base. The compounds of invention may be sufficiently basic or acidic to form stable nontoxic acid or base salts, administration of the compound as a pharmaceutically acceptable salt may be appropriate. Non-limiting Examples of pharmaceutically acceptable salts are inorganic, organic acid addition salts formed by addition of acids including hydrochloride salts. Non-limiting Examples of pharmaceutically acceptable salts are inorganic, organic base addition salts formed by addition of bases. The compounds of the invention may also form salts with amino acids. Pharmaceutically acceptable salts may be obtained using standard procedures well known in the art, for example by reacting sufficiently basic compound such as an amine with a suitable acid.

Screening of the compounds of invention for antiviral activity, particularly against coronavirus can be achieved by using various in-vitro mentioned herein below or methods known in the art.

### Pharmaceutical Compositions

The invention relates to pharmaceutical compositions containing the compounds of the formula (I), stereoisomers or pharmaceutically acceptable salts thereof disclosed herein. In particular, pharmaceutical compositions containing a therapeutically effective amount of at least one compound of formula (I) described herein and at least one pharmaceutically acceptable excipient (such as a carrier or diluent). Preferably, the contemplated pharmaceutical compositions include the compound(s) described herein in an amount sufficient to treat the viral infections described herein when administered to a subject.

The subjects contemplated include, for example, a living cell and a mammal, including human. The compound of the invention may be associated with a pharmaceutically acceptable excipient (such as a carrier or a diluent) or be diluted by a carrier, or enclosed within a carrier which can be in the form of a capsule, sachet, paper or other container. The pharmaceutically acceptable excipient includes pharmaceutical agent that does not itself induce the production of antibodies harmful to the individual receiving the composition, and which may be administered without undue toxicity.

Examples of suitable carriers or excipients include, but are not limited to, water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, salicylic acid, fatty acids, fatty acid amines, fatty acid monoglycerides and diglycerides, pentaerythritol fatty acid esters, polyoxyethylene, hydroxymethylcellulose and polyvinylpyrrolidone.

The pharmaceutical composition may also include one or more pharmaceutically acceptable auxiliary agents, wetting agents, emulsifying agents, suspending agents, preserving agents, salts for influencing osmotic pressure, buffers, sweetening agents, flavoring agents, colorants, or any combination of the foregoing. The pharmaceutical composition of the invention may be formulated so as to provide quick, sustained, or delayed release of the active ingredient after administration to the subject by employing procedures known in the art.

The pharmaceutical compositions described herein may be prepared by conventional techniques known in the art. For example, the active compound can be mixed with a carrier, or diluted by a carrier, or enclosed within a carrier, which may be in the form of an ampoule, capsule, sachet, paper, or other container. When the carrier serves as a diluent, it may be a solid, semi-solid, or liquid material that acts as a vehicle, excipient, or medium for the active compound. The active compound can be adsorbed on a granular solid container, for Example, in a sachet. The pharmaceutical compositions may be in conventional forms, for example, capsules, tablets, caplets, orally disintegrating tablets, aerosols, solutions, suspensions or products for topical application.

The route of administration may be any route which effectively transports the active compound of the invention to the appropriate or desired site of action. Suitable routes of administration include, but are not limited to, oral, oral inhalation, nasal, pulmonary, buccal, subdermal, intradermal, transdermal, parenteral, rectal, depot, subcutaneous, intravenous, intraurethral, intramuscular, intranasal, ophthalmic (such as with an ophthalmic solution) or topical (such as with a topical ointment).

Solid oral formulations include, but are not limited to, tablets, caplets, capsules (soft or hard gelatin), orally disintegrating tablets, dragees (containing the active ingredient in powder or pellet form), troches and lozenges. Tablets, dragees, or capsules having talc and/or a carbohydrate carrier or binder or the like are particularly suitable for oral application. Liquid formulations include, but are not limited to, syrups, emulsions, suspensions, solutions, soft gelatin and sterile injectable liquids, such as aqueous or non-aqueous liquid suspensions or solutions. For parenteral application, particularly suitable are injectable solutions or suspensions, preferably aqueous solutions with the active compound dissolved in polyhydroxylated castor oil.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as pocketed tablets, capsules, and powders in vials or ampoules. Also, the unit dosage form can be a capsule, tablet, caplet, cachet, or lozenge itself, or it can be the appropriate number of any of these in packaged form.

For administration to subject patients, the total daily dose of the compounds of the invention depends, of course, on the mode of administration. For example, oral administration may require a higher total daily dose, than an intravenous (direct into blood). The quantity of active component in a unit dose preparation may be varied or adjusted from 0.1 mg to 1000 mg by oral administration and 1 pg to 5000 pg by inhalation according to the potency of the active component or mode of administration.

Those skilled in the relevant art can determine suitable doses of the compounds for use in treating the diseases and disorders described herein. Therapeutic doses are generally identified through a dose ranging study in subject based on preliminary evidence derived from the animal studies. Doses must be sufficient to result in a desired therapeutic benefit without causing unwanted side effects for the patient. For example, the daily dosage of antiviral can range from about 0.1 to about 30.0 mg/kg by oral administration. Mode of administration, dosage forms, suitable pharmaceutical excipients, diluents or carriers can also be well used and adjusted by those skilled in the art. All changes and modifications envisioned are within the scope of the invention.

In another aspect, the invention relates to a method of treating diseases, disorders or conditions associated with coronavirus infections caused by SARS, MERS, SARS-COV-2 and other viral diseases. In this method, a subject in need of such treatment is administered a therapeutically effective amount of a compound of formula (I) or a pharmaceutically acceptable salt thereof as described herein.

It is to be understood that the invention encompasses the compounds of formula (I), stereoisomers or pharmaceutically acceptable salts thereof in the manufacture of a medicament for treating a disease or disorder mentioned herein. Standard experimental procedures are followed for the synthesis of benzimidazole compounds. Chemicals and solvents were generally used from common suppliers and were used further without purification. Silica gel 60 F254 analytical thin layer chromatography (TLC) plates were used from Merck and visualized under UV light and/or with potassium permanganate stain and/or with ninhydrin stain and/or with Iodine. Chromatographic purifications were performed using Biotage flash column chromatography system. Deuterated solvents to record NMR spectra were purchased from Sigma-Aldrich. All ¹H NMR spectra were recorded using 500 MHz NMR spectrometer. Chemical shifts (δ) and coupling constants (J) are given in ppm and Hz respectively. Mass spectrometry analysis (m/z) and HPLC were performed utilising Agilent LCMS (1290 LC/MSD) single quad system.

The general synthetic approach for synthesis of compounds related to scaffold (I) [wherein R₁, R₂, R₃, R₄ and n are as defined with respect to a compound of formula (I)] is depicted below as synthetic scheme 1.

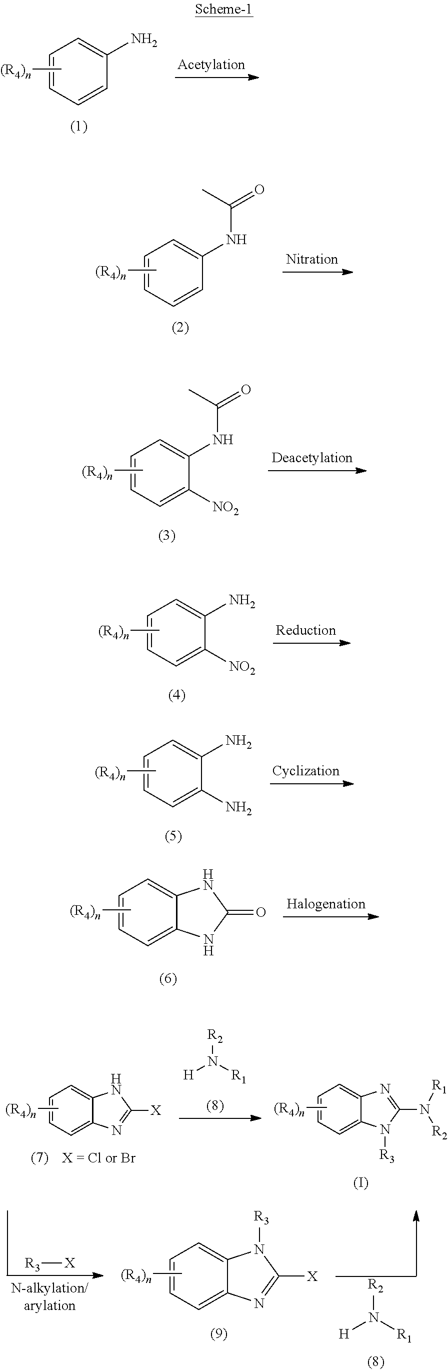

The compound of formula (I) can be prepared starting from commercially available substituted aniline of formula (1). This substituted aniline may undergo acetylation in the presence of suitable anhydride such as acetic anhydride to give compound of formula (2). The compound of formula (2) can undergo nitration to give compound of formula (3). Further, this acetamide of formula (3) treated with a base such as sodium hydroxide, potassium hydroxide etc. can afford compound of formula (4). The nitro group of compounds of formula (4) may reduce to diamine of the formula (5) using reducing agent such as H₂, Pd/C, Iron in acetic acid, SnCl₂ etc. Further, the cyclization reaction of diamine of formula (5) with coupling agents such as carbonyldiimidazole, triphosgene, etc in the presence or absence of catalytic amount of a base like DMAP may afford compound of formula (6). Using an appropriate halogenating agent such as phosphoryl chloride/bromide may afford compound of formula (7). Nucleophilic substitution of compound of formula (7) with a suitable aniline or amine (8) in acidic conditions using acids such as HCl afford compounds of Formula (I). The N-substitution of compound of formula (7) using alkylating/arylating agent in basic condition may form compound of formula (9) which on nucleophilic substitution with suitable aniline/amine of formula (8) affords compound of formula (I). The compounds described herein including compounds of general formula (I) can be prepared by using techniques known to one skilled in the art through the reaction sequences described in the scheme 1 as well as by other methods. Further, in the following schemes, where specific bases, acids, reagents, solvents, etc are mentioned, it is understood that other bases, acids, reagents, solvents, etc known in the art may also be used and are therefore included within the scope of the present invention. Variations in reaction conditions, for example, temperature and/or duration of the reaction, which may be used as known in the art, are also within the scope of the present invention. All the isomers of the compound of formula are described in these schemes, unless otherwise specified, are also encompassed within the scope of this invention. Unless otherwise stated, work-up implies the following operations: distribution of the reaction mixture between the aqueous and organic phase, separation of layers, drying the organic layer over sodium sulphate or magnesium sulphate, filtration and evaporation of the organic solvent. Purification, unless otherwise mentioned, implies crystallization or purification by silica gel/reverse phase chromatographic techniques, generally using ethyl acetate/petroleum ether, methanol/dichloromethane, or acetonitrile/water mixture of a suitable polarity as the mobile phase.

The intermediates and the compounds of the present invention may be obtained in pure form in a manner known per se, for example, by distilling off the solvent in vacuum and re-crystallizing the residue obtained from a suitable solvent, such as pentane, diethyl ether, isopropyl ether, chloroform, dichloromethane, ethyl acetate, acetone or their combinations or subjecting it to one of the purification methods, such as column chromatography (e.g., flash chromatography) on a suitable support material such as alumina or silica gel using eluent such as dichloromethane, ethyl acetate, hexane, methanol, acetone and their combinations in normal phase or acetonitrile, water and methanol combinations in reverse phase. Preparative LC-MS (liquid chromatography-mass spectrometry) method is also used for the purification of molecules described herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### General Procedure:

The intermediates described below were prepared using synthetic scheme depicted above.

### Intermediate 1: 2-chloro-4,6-bis(trifluoromethyl)-1H-benzo[d]imidazole

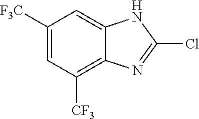 [SMILES: FC(F)(F)c1cc(C(F)(F)F)c2nc(Cl)[nH]c2c1]

### Step 1: Preparation of N-(3,5-bis(trifluoromethyl)phenyl)acetamide

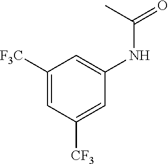 [SMILES: CC(=O)Nc1cc(C(F)(F)F)cc(C(F)(F)F)c1]

A mixture of compound 3,5-bis(trifluoromethyl)aniline (3 g, 13.1 mmol) and acetic anhydride (15 mL) was stirred at 35° C. for 3 h. The reaction mixture was concentrated in vacuum to yield the title compound as a white solid (3.36 g, 94.64%).

LCMS (ESI): m/z=272.0 (M+H)⁺

¹H NMR (500 MHz, DMSO-d6): δ 10.56 (bs, 1H), 8.22 (s, 2H), 7.65 (s, 1H), 2.11 (s, 3H).

### Step 2: Preparation of N-(2-nitro-3,5-bis(trifluoromethyl)phenyl)acetamide

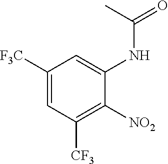 [SMILES: CC(=O)Nc1cc(C(F)(F)F)cc(C(F)(F)F)c1[N+](=O)[O-]]

To a stirred solution of N-(3,5-bis(trifluoromethyl)phenyl)acetamide (3.36 g, 12.4 mmol) in conc. H₂SO₄ (sulphuric acid, 8 mL), 70% HNO₃ (nitric acid, 2.5 mL) was added in a dropwise manner at −10° C. The resultant mixture was stirred at 35° C. for 3 h. The reaction mixture was poured onto crushed ice and carefully neutralized with solid NaHCO₃ (sodium bicarbonate) and extracted with ethyl acetate. The combined organic extracts were washed with brine and dried over Na₂SO₄ (sodium sulphate), filtered and dried in vacuo. The crude compound was purified by flash column chromatography (Biotage) using eluent (1:10; ethyl acetate:n-hexane) to get the title compound as a white solid (1.92 g, 47.76%).

LCMS (ESI): m/z=317.0 (M+H)⁺

¹H NMR (500 MHz, DMSO-d6): δ 10.49 (bs, 1H), 8.45 (d, J=1.1 Hz, 1H), 8.19 (s, 1H), 2.10 (s, 3H).

### Step 3: Preparation of 2-nitro-3,5-bis(trifluoromethyl)aniline

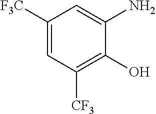 [SMILES: Nc1cc(C(F)(F)F)cc(C(F)(F)F)c1O]

To a solution of N-(2-nitro-3,5-bis(trifluoromethyl)phenyl)acetamide (1.92 g, 6.1 mmol) in MeOH (methanol, 4 mL), aqueous 3N NaOH (sodium hydroxide, 20 mL) was added at room temperature. The reaction mixture was allowed to stir at 110° C. for 2 h, then cooled to room temperature. The reaction mixture was extracted with ethyl acetate (3×100 mL), washed with brine, organic layer was dried over Na₂SO₄, filtered and concentrated to get the title compound, which was used immediately in the next step.

LCMS (ESI): m/z=272.9 (M−H)⁻

¹H NMR (500 MHz, DMSO-d6): δ 7.60 (s, 1H), 7.26 (s, 1H), 6.91 (bs, 2H).

### Step 4: Preparation of 3,5-bis(trifluoromethyl)benzene-1,2-diamine

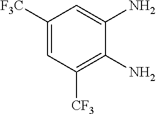 [SMILES: Nc1cc(C(F)(F)F)cc(C(F)(F)F)c1N]

To a solution of 2-nitro-3,5-bis(trifluoromethyl)aniline (1.5 g, 5.47 mmol) in EtOH (ethanol) was added 10% Pd/C (palladium on carbon, 50 mg) under nitrogen atmosphere. The reaction vessel was charged with hydrogen gas and stirred at room temperature for overnight. After completion of the reaction, monitored by TLC analysis, the vessel contents were filtered through a short pad of celite. The celite pad was washed with EtOAc (ethyl acetate) and the filtrate was concentrated in vacuo. The crude compound was purified by flash column chromatography (Biotage) using eluent (1:20, ethyl acetate:n-hexane) to get the title compound as a pale-yellow oil (1.32 g, 88.43%).

LCMS (ESI): m/z=245.0 (M+H)⁺

¹H NMR (500 MHz, DMSO-d6): δ 7.00 (d, J=1.9 Hz, 1H), 6.92 (s, 1H), 5.57 (bs, 2H), 5.39 (bs, 2H).

### Step 5: Preparation of 4,6-bis(trifluoromethyl)-1,3-dihydro-2H-benzo[d]imidazol-2-one

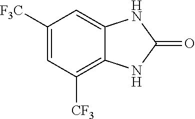 [SMILES: O=c1[nH]c2cc(C(F)(F)F)cc(C(F)(F)F)c2[nH]1]

To a solution of 3,5-bis(trifluoromethyl)benzene-1,2-diamine (1.32 g, 5.41 mmol) in THF (tetrahydrofuran), was added 1,1′-carbonylbis-1H-imidazole (carbonyldiimidazole, 3.5 g, 21.63 mmol). The reaction mixture was stirred at 35° C. for 4 h, after which the vessel contents were concentrated in vacuo. The crude compound was purified by flash column chromatography (Biotage) using eluent (1:4, ethyl acetate:n-hexane) to get the title compound as a white solid (1.32 g, 91%).

LCMS (ESI): m/z=269.0 (M−H)⁻

¹H NMR (500 MHz, MeOD-d4): δ 7.44 (s, 1H), 7.40 (s, 1H).

### Step 6: Preparation of 2-chloro-4,6-bis(trifluoromethyl)-1H-benzo[d]imidazole

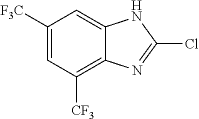 [SMILES: FC(F)(F)c1cc(C(F)(F)F)c2nc(Cl)[nH]c2c1]

A solution of 4,6-bis(trifluoromethyl)-1,3-dihydro-2H-benzo[d]imidazol-2-one (1.3 g, 4.8 mmol) in POCl₃ (Phosphoryl chloride, 25 mL) was refluxed for 17 h. The reaction mixture was concentrated in vacuo, neutralized using saturated NaHCO₃, extracted with EtOAc, washed with water, brine and dried over Na₂SO₄ filtered and concentrated in vacuo. The crude compound was purified by flash column chromatography (Biotage) using eluent (1:12, ethyl acetate:n-hexane) to get the intermediate (1) as a white solid (0.47 g, 34%).

LCMS (ESI): m/z=288.6; 290.6 (M+H)⁺

¹H NMR (500 MHz, MeOD-d4): δ 8.00 (s, 1H), 7.72 (s, 1H).

The following intermediates were synthesized using a sequence analogous to that described for intermediate 1:

| | | | m/z | |
|---|---|---|---|---|
| Int. | | | (ESI) | |
| No. | Structure | IUPAC name | (M − H)⁻ | NMR data |
| 2 | 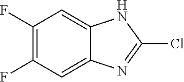 [SMILES: Fc1cc2nc(Cl)[nH]c2cc1F] | 2-chloro-5,6-difluoro- 1H- benzo[d]imidazole | 186.9; 188.9 | ¹H NMR (500 MHz, DMSO-d6): δ 13.53 (bs, 1H), 7.62 (m, 2H). |
| 3 | 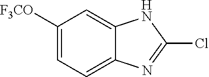 [SMILES: FC(F)(F)Oc1ccc2nc(Cl)[nH]c2c1] | 2-chloro-6- (trifluoromethoxy)- 1H- benzo[d]imidazole | 234.9; 236.9 | ¹H NMR (500 MHz, DMSO-d6): δ 13.57 (bs, 1H), 7.61 (d, J = 8.6 Hz, 1H), 7.55 (s, 1H), 7.23 (dd, J = 8.8, 1.2 Hz, 1H). |
| 4 | 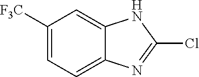 [SMILES: FC(F)(F)c1ccc2nc(Cl)[nH]c2c1] | 2-chloro-6- (trifluoromethyl)-1H- benzo[d]imidazole | 218.9; 220.9 | ¹H NMR (500 MHz, DMSO-d6): δ 13.76 (bs, 1H), 8.07 - 7.62 (m, 2H), 7.57 (d, J = 8.2 Hz, 1H). |
| 5 | 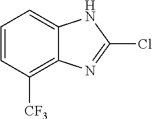 [SMILES: FC(F)(F)c1cccc2[nH]c(Cl)nc12] | 2-chloro-4- (trifluoromethyl)-1H- benzo[d]imidazole | 218.9; 220.9 | ¹H NMR (500 MHz, DMSO-d6): δ 13.81 (bs, 1H), 7.80 (d, J = 5.8 Hz, 1H), 7.56 (d, J = 7.6 Hz, 1H), 7.43- 7.36 (m, 1H). |
| 6 | 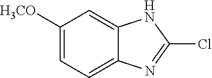 [SMILES: COc1ccc2nc(Cl)[nH]c2c1] | 2-chloro-6-methoxy- 1H- benzo[d]imidazole | 181.0; 182.9 | ¹H NMR (500 MHz, DMSO-d6): δ 13.04 (bs, 1H), 7.40 (d, J = 6.7 Hz, 1H), 7.00 (s, 1H), 6.84 (dd, J = 8.8, 2.3 Hz, 1H). |

### Intermediate 7: 2-chloro-5,6-difluoro-1-methyl-1H-benzo[d]imidazole

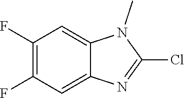

Into a stirred solution of 2-chloro-5,6-difluoro-1H-benzo[d]imidazole (100 mg, 0.53 mmol) in DMF, NaH (21 mg, 0.53 mmol) was added at 0° C. After 30 min of stirring, methyl iodide was added into the reaction mixture. This mixture was then stirred at 35° C. for overnight. After completion of reaction, the reaction mixture was neutralized with NH₄Cl, extracted with ethyl acetate, washed with water, brine and dried over Na₂SO₄ filtered and concentrated in vacuo. The crude compound was purified by flash column chromatography (Biotage) using eluent (1:10, ethyl acetate:n-hexane) to get the intermediate (7) as a white solid (100 mg, 93.34%).

LCMS (ESI): m/z=203.0; 205.0 (M+H)⁺

¹H NMR (500 MHz, DMSO-d6): δ 7.84 (dd, J=10.7, 7.3 Hz, 1H), 7.71 (dd, J=10.9, 7.4 Hz, 1H), 3.33 (s, 3H).

### Synthesis of Example Compounds

### Example 1: N-(3,5-bis(trifluoromethyl)phenyl)-4,6-bis(trifluoromethyl)-1H-benzo[d]imidazol-2-amine

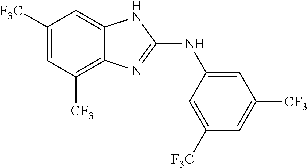 [SMILES: FC(F)(F)c1cc(Nc2nc3c(C(F)(F)F)cc(C(F)(F)F)cc3[nH]2)cc(C(F)(F)F)c1]

To a stirred solution of 2-chloro-4,6-bis(trifluoromethyl)-1H-benzo[d]imidazole (Intermediate-1) (50 mg, 0.174 mmol) in n-butanol (3 mL) was added 3,5-bis(trifluoromethyl)aniline (54 μL, 0.347 mmol) and catalytic 30% aq. HCl. The reaction mixture was refluxed for 24 h. After the completion of the reaction, monitored by TLC analysis, the reaction mixture was diluted with EtOAc and washed with sat. aq. NaHCO₃, brine, dried over Na₂SO₄ and concentrated in vacuo. The crude compound was purified by flash column chromatography (Biotage) using eluent (1:20, ethyl acetate:n-hexane) to get the title compound as off-white solid (20 mg).

LCMS (ESI): m/z=482.0 (M+H)⁺

¹H NMR (500 MHz, DMSO-d6): δ 11.11 (bs, 1H), 8.63 (s, 2H), 7.94 (s, 1H), 7.64 (m, 2H).

### Example 2: N-(4-chlorophenyl)-4,6-bis(trifluoromethyl)-1H-benzo[d]imidazol-2-amine

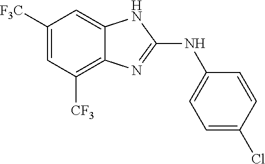 [SMILES: FC(F)(F)c1cc(C(F)(F)F)c2nc(Nc3ccc(Cl)cc3)[nH]c2c1]

Following a procedure analogous to the one provided for compound of example 1 but 4-chloroaniline was used instead of 3,5-bis(trifluoromethyl)aniline in this case to obtain the title compound as off-white solid.

LCMS (ESI): m/z=380.0 (M+H)⁺

¹H NMR (500 MHz, MeOD-d4): δ 7.83 (s, 1H), 7.74 (s, 1H), 7.64-7.60 (m, 2H), 7.49-7.45 (m, 2H).

### Example 3: N-phenyl-4,6-bis(trifluoromethyl)-1H-benzo[d]imidazol-2-amine

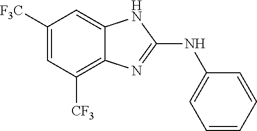 [SMILES: FC(F)(F)c1cc(C(F)(F)F)c2nc(Nc3ccccc3)[nH]c2c1]

Following a procedure analogous to the one provided for compound of example 1 but aniline was used instead of 3,5-bis(trifluoromethyl)aniline in this case to obtain the title compound as off-white solid.

LCMS (ESI): m/z=346.0 (M+H)⁺

¹H NMR (500 MHz, MeOD-d4): δ 7.86 (s, 1H), 7.83 (s, 1H), 7.58-7.52 (m, 4H), 7.42-7.37 (m, 1H).

### Example 4: N-(3-bromo-4-methoxyphenyl)-4,6-bis(trifluoromethyl)-1H-benzo[d]imidazol-2-amine

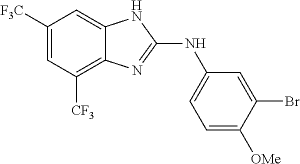 [SMILES: COc1ccc(Nc2nc3c(C(F)(F)F)cc(C(F)(F)F)cc3[nH]2)cc1Br]

Following a procedure analogous to the one provided for compound of example 1 but 3-bromo-4-methoxyaniline was used instead of 3,5-bis(trifluoromethyl)aniline in this case to obtain the title compound as off-white solid.

LCMS (ESI): m/z=453.9; 455.9 (M+H)⁺

¹H NMR (500 MHz, MeOD-d4): δ 7.82 (d, J=2.6 Hz, 2H), 7.79 (s, 1H), 7.53 (dd, J=8.8, 2.6 Hz, 1H), 7.18 (d, J=8.8 Hz, 1H), 3.94 (s, 3H).

### Example 5: N-(pyridin-3-yl)-4,6-bis(trifluoromethyl)-1H-benzo[d]imidazol-2-amine

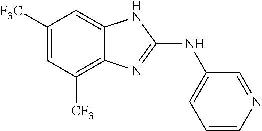 [SMILES: FC(F)(F)c1cc(C(F)(F)F)c2nc(Nc3cccnc3)[nH]c2c1]

Following a procedure analogous to the one provided for compound of example 1 but 3-aminopyridine was used instead of 3,5-bis(trifluoromethyl)aniline in this case to obtain the title compound as a brown solid.

LCMS (ESI): m/z=347.0 (M+H)⁺

¹H NMR (500 MHz, MeOD-d4): δ 8.87 (bs, 1H), 8.69 (bs, 1H), 8.23 (s, 1H), 7.91-7.80 (m, 4H), 7.62-7.56 (m, 1H).

### Example 6: N-benzyl-4,6-bis(trifluoromethyl)-1H-benzo[d]imidazol-2-amine

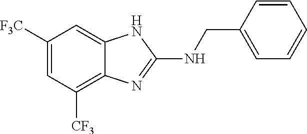 [SMILES: FC(F)(F)c1cc(C(F)(F)F)c2nc(NCc3ccccc3)[nH]c2c1]

Following a procedure analogous to the one provided for compound of example 1 but benzylamine was used instead of 3,5-bis(trifluoromethyl)aniline in this case to obtain the title compound as off-white solid.

LCMS (ESI): m/z=360.1 (M+H)⁺

¹H NMR (500 MHz, MeOD-d4): δ 7.90 (s, 1H), 7.88 (s, 1H), 7.49-7.40 (m, 4H), 7.39-7.33 (m, 1H), 4.79 (s, 2H).

### Example 7: N-(tetrahydro-2H-pyran-4-yl)-4,6-bis(trifluoromethyl)-1H-benzo[d]imidazol-2-amine

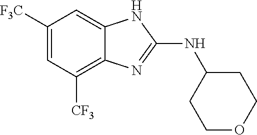 [SMILES: FC(F)(F)c1cc(C(F)(F)F)c2nc(NC3CCOCC3)[nH]c2c1]

Following a procedure analogous to the one provided for compound of example 1 but 4-aminotetrahydropyran was used instead of 3,5-bis(trifluoromethyl)aniline in this case to obtain the title compound.

LCMS (ESI): m/z=354.1 (M+H)⁺

¹H NMR (500 MHz, DMSO-d6): δ 7.80 (s, 1H), 7.69 (s, 1H), 3.91 (dt, J=11.5, 3.4 Hz, 2H), 3.44 (td, J=11.5, 2.0 Hz, 2H), 2.52-2.49 (m, 1H), 1.95 (dd, J=12.4, 2.1 Hz, 2H), 1.66-1.55 (m, 2H).

### Example 8: 5,6-difluoro-N-phenyl-1H-benzo[d]imidazol-2-amine

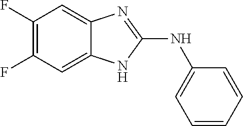 [SMILES: Fc1cc2nc(Nc3ccccc3)[nH]c2cc1F]

Following a procedure analogous to the one provided for compound of example 1 but replacing 3,5-bis(trifluoromethyl)aniline with aniline and intermediate 1 with intermediate 2 to obtain the title compound.

LCMS (ESI): m/z=246.0 (M+H)⁺

¹H NMR (500 MHz, DMSO-d6): δ 11.00 (bs, 1H), 9.51 (bs, 1H), 7.72 (d, J=7.6 Hz, 2H), 7.40-7.23 (m, 4H), 6.97-6.91 (m, 1H).

### Example 9: N-(3-bromo-4-methoxyphenyl)-5,6-difluoro-1H-benzo[d]imidazol-2-amine

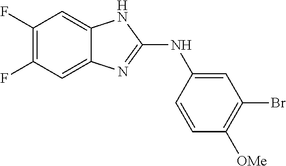 [SMILES: COc1ccc(Nc2nc3cc(F)c(F)cc3[nH]2)cc1Br]

To a stirred solution of Intermediate (2) (45 mg, 0.53 mmol) in n-butanol (2 mL) was added 3-bromo-4-methoxyaniline (214 mg, 1 mmol) and catalytic 30% aq. HCl. The reaction mixture was stirred at 175° C. under microwave irradiation for 2 h. After the completion of the reaction, monitored by TLC analysis, the reaction mixture was diluted with EtOAc and washed with sat. aq. NaHCO₃, brine, dried over Na₂SO₄ and concentrated in vacuo. The crude compound was purified by flash column chromatography (Biotage) using eluent (1:5, ethyl acetate:n-hexane) to get the title compound as an off-white solid (66 mg).

LCMS (ESI): m/z=354.0, 356.0 (M+H)⁺

¹H NMR (500 MHz, MeOD-d4): δ 7.66 (d, J=2.6 Hz, 1H), 7.38 (dd, J=8.8, 2.6 Hz, 1H), 7.29 (t, J=8.3 Hz, 2H), 7.16 (d, J=8.8 Hz, 1H), 3.90 (s, 3H).

### Example 10: 5,6-difluoro-N-(3-(trifluoromethyl)phenyl)-1H-benzo[d]imidazol-2-amine

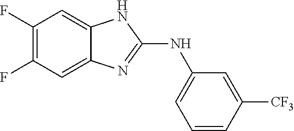 [SMILES: Fc1cc2nc(Nc3cccc(C(F)(F)F)c3)[nH]c2cc1F]

Following a procedure analogous to the one provided for compound of example 9 but 3-aminobenzotrifluoride was used instead of 3-bromo-4-methoxyaniline to obtain the title compound as off-white solid.

LCMS (ESI): m/z=314.0 (M+H)+

¹H NMR (500 MHz, DMSO-d6): δ 7.88 (m, 2H), 7.65 (t, J=7.9 Hz, 1H), 7.47 (m, 3H).

### Example 11: N-(3,5-bis(trifluoromethyl)phenyl)-5,6-difluoro-1H-benzo[d]imidazol-2-amine

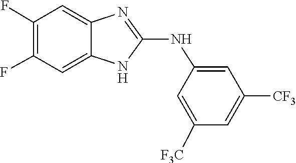 [SMILES: Fc1cc2nc(Nc3cc(C(F)(F)F)cc(C(F)(F)F)c3)[nH]c2cc1F]

Following a procedure analogous to the one provided for compound of example 9 but 3-bromo-4-methoxyaniline replaced with 3,5-bis(trifluoromethyl)aniline in this case to obtain the title compound as off-white solid.

LCMS (ESI): m/z=382.0 (M+H)⁺

¹H NMR (500 MHz, DMSO-d6): δ 10.5 (bs, 1H), 8.46 (s, 2H), 7.61 (s, 1H), 7.43 (t, J=9.0 Hz, 2H).

### Example 12: 5,6-difluoro-N-(4-(trifluoromethyl)phenyl)-1H-benzo[d]imidazol-2-amine

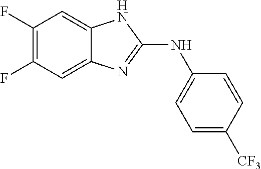 [SMILES: Fc1cc2nc(Nc3ccc(C(F)(F)F)cc3)[nH]c2cc1F]

Following a procedure analogous to the one provided for compound of example 9 but 3-bromo-4-methoxyaniline replaced with 4-aminobenzotrifluoride in this case to obtain the title compound as off-white solid.

LCMS (ESI): m/z=314.0 (M+H)⁺

¹H NMR (500 MHz, DMSO-d6): δ 11.24 (bs, 1H), 10.03 (bs, 1H), 7.94 (d, J=8.5 Hz, 2H), 7.67 (d, J=8.6 Hz, 2H), 7.44 (dd, J=11.2, 7.5 Hz, 1H), 7.32 (dd, J=10.3, 7.6 Hz, 1H).

### Example 13: N-benzyl-5,6-difluoro-1H-benzo[d]imidazol-2-amine

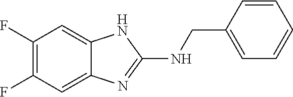 [SMILES: Fc1cc2nc(NCc3ccccc3)[nH]c2cc1F]

Following a procedure analogous to the one provided for compound of example 9, 3-bromo-4-methoxyaniline replaced with benzyl amine in this case to obtain the title compound as off-white solid.

LCMS (ESI): m/z=260.0 (M+H)⁺

¹H NMR (500 MHz, DMSO-d6): δ 13.19 (bs, 1H), 9.69 (bs, 111), 7.51-7.43 (m, 4H), 7.40 (t, J=7.5 Hz, 2H), 7.33 (t, J=7.2 Hz, 1H), 4.67 (d, J=6.1 Hz, 2H).

### Example 14: 4,6-bis(trifluoromethyl)-N-(3-(trifluoromethyl)phenyl)-1H-benzo[d]imidazol-2-amine

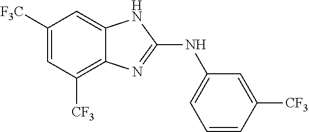 [SMILES: FC(F)(F)c1cccc(Nc2nc3c(C(F)(F)F)cc(C(F)(F)F)cc3[nH]2)c1]

Following a procedure analogous to the one provided for compound of example 9 but 3-aminobenzotrifluoride and intermediate-1 was used in this case to obtain the title compound as off-white solid.

LCMS (ESI): m/z=414.0 (M+H)⁺

¹H NMR (500 MHz, MeOD-d4): δ 8.19 (s, 1H), 7.91 (d, J=8.2 Hz, 1H), 7.81 (s, 1H), 7.58 (s, 1H), 7.52 (t, J=8.0 Hz, 1H), 7.31 (d, J=7.7 Hz, 1H).

### Example 15: N-isopropyl-4,6-bis(trifluoromethyl)-1H-benzo[d]imidazol-2-amine

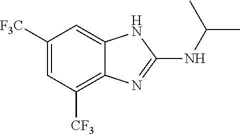 [SMILES: CC(C)Nc1nc2c(C(F)(F)F)cc(C(F)(F)F)cc2[nH]1]

Following a procedure analogous to the one provided for compound of example 9 but propan-2-amine and intermediate-1 was used in this case to obtain the title compound as off-white solid.

LCMS (ESI): m/z=312.3 (M+H)⁺

¹H NMR (500 MHz, MeOD-d4): δ 7.52 (s, 1H), 7.36 (s, 1H), 3.95 (m, 1H), 1.21 (d, J=6.5 Hz, 6H).

### Example 16: 2-(piperidin-1-yl)-4,6-bis(trifluoromethyl)-1H-benzo[d]imidazole

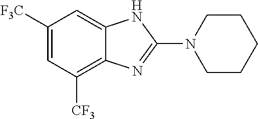 [SMILES: FC(F)(F)c1cc(C(F)(F)F)c2nc(N3CCCCC3)[nH]c2c1]

Following a procedure analogous to the one provided for compound of example 9 but piperidine and intermediate-1 was used in this case to obtain the title compound as off-white solid.

LCMS (ESI): m/z=338.1 (M+H)⁺

¹H NMR (500 MHz, MeOD-d4): δ 7.58 (s, 1H), 7.48 (s, 1H), 3.66 (m, 4H), 1.71 (m, 6H).

### Example 17: N-(naphthalen-2-yl)-5,7-bis(trifluoromethyl)-1H-benzo[d]imidazol-2-amine

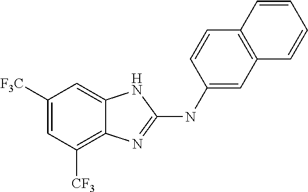 [SMILES: FC(F)(F)c1cc(C(F)(F)F)c2nc(Nc3ccc4ccccc4c3)[nH]c2c1]

Following a procedure analogous to the one provided for compound of example 9 but naphthalen-2-amine and intermediate-1 was used in this case to obtain the title compound as off-white solid.

LCMS (ESI): m/z=396.0 (M+H)⁺

¹H NMR (500 MHz, MeOD-d4): δ 8.14 (d, J=2.1 Hz, 1H), 8.06 (d, J=8.8 Hz, 1H), 7.99-7.92 (m, 2H), 7.89 (s, 1H), 7.86 (s, 1H), 7.63 (dd, J=8.7, 2.2 Hz, 1H), 7.58 (m, 2H).

### Example 18: N-(2-isopropylphenyl)-4,6-bis(trifluoromethyl)-1H-benzo[d]imidazol-2-amine

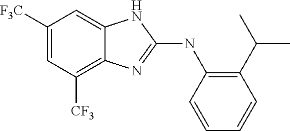 [SMILES: CC(C)c1ccccc1Nc1nc2c(C(F)(F)F)cc(C(F)(F)F)cc2[nH]1]

Following a procedure analogous to the one provided for compound of example 9 but 2-isopropylaniline and intermediate-1 was used in this case to obtain the title compound as off-white solid.

LCMS (ESI): m/z=388.1 (M+H)⁺

¹H NMR (500 MHz, MeOD-d4): δ 7.84 (s, 1H), 7.80 (s, 1H), 7.55 (dd, J=7.9, 1.5 Hz, 1H), 7.50 (td, J=7.6, 1.3 Hz, 1H), 7.45 (dd, J=7.9, 1.3 Hz, 1H), 7.38 (m, 1H), 3.46-3.16 (m, 1H), 1.24 (d, J=6.9 Hz, 6H).

### Example 19: (R)—N-(1-(naphthalen-1-yl)ethyl)-4,6-bis(trifluoromethyl)-1H-benzo[d]imidazol-2-amine

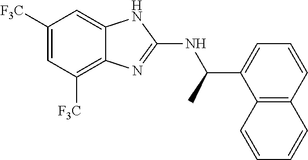 [SMILES: C[C@@H](Nc1nc2c(C(F)(F)F)cc(C(F)(F)F)cc2[nH]1)c1cccc2ccccc12]

Following a procedure analogous to the one provided for compound of example 9 but (R)-1-(naphthalen-1-yl)ethan-1-amine and intermediate-1 was used in this case to obtain the title compound as off-white solid.

LCMS (ESI): m/z=424.1 (M+H)⁺

¹H NMR (500 MHz, DMSO-d6): δ 11.45 (bs, 1H), 8.38 (bs, 1H), 8.27 (d, J=8.3 Hz, 1H), 7.96 (dd, J=8.2, 0.8 Hz, 1H), 7.85 (d, J=8.2 Hz, 1H), 7.67 (d, J=7.0 Hz, 1H), 7.57 (m, 3H), 7.53-7.49 (m, 1H), 7.46 (s, 1H), 5.83 (s, 1H), 1.68 (d, J=6.8 Hz, 3H).

### Example 20: 3-((4,6-bis(trifluoromethyl)-1H-benzo[d]imidazol-2-yl)amino)tetrahydrothiophene 1,1-dioxide

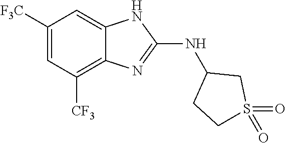 [SMILES: O=S1(=O)CCC(Nc2nc3c(C(F)(F)F)cc(C(F)(F)F)cc3[nH]2)C1]

Following a procedure analogous to the one provided for compound of example 9 but 3-aminotetrahydrothiophene 1,1-dioxide and intermediate-1 was used in this case to obtain the title compound as off-white solid.

LCMS (ESI): m/z=388.0 (M+H)⁺

¹H NMR (500 MHz, MeOD-d4): δ 7.68 (s, 1H), 7.51 (s, 1H), 4.81-4.74 (bs, 1H), 3.64 (dd, J=13.6, 7.4 Hz, 1H), 3.37 (m, 1H), 3.27-3.14 (m, 2H), 2.76-2.62 (m, 1H), 2.44-2.31 (m, 1H).

### Example 21: N-(1-methylcyclobutyl)-4,6-bis(trifluoromethyl)-1H-benzo[d]imidazol-2-amine

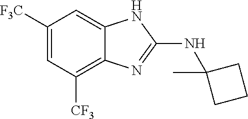 [SMILES: CC1(Nc2nc3c(C(F)(F)F)cc(C(F)(F)F)cc3[nH]2)CCC1]

Following a procedure analogous to the one provided for the compound of example 9 but 1-methylcyclobutan-1-amine and intermediate-1 were used in this case to obtain the title compound as off-white solid.

LCMS (ESI): m/z=338.1 (M+H)⁺

¹H NMR (500 MHz, MeOD-d4): δ 8.10 (bs, 1H), 7.82 (s, 1H), 7.66 (s, 1H), 7.45 (bs, 1H), 2.46-2.37 (m, 2H), 2.23-2.15 (m, 2H), 2.01-1.87 (m, 2H), 1.60 (s, 3H).

### Example 22: N-(3-(trifluoromethoxy)phenyl)-4,6-bis(trifluoromethyl)-1H-benzo[d]imidazol-2-amine

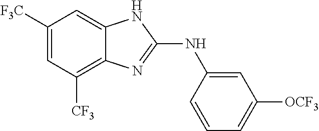 [SMILES: FC(F)(F)Oc1cccc(Nc2nc3c(C(F)(F)F)cc(C(F)(F)F)cc3[nH]2)c1]

Following a procedure analogous to the one provided for compound of example 9 but 3-(trifluoromethoxy)aniline and intermediate-1 was used in this case to obtain the title compound as off-white solid.

LCMS (ESI): m/z=430.0 (M+H)⁺

¹H NMR (500 MHz, MeOD-d4): δ 8.04 (bs, 1H), 7.77 (s, 1H), 7.63 (s, 1H), 7.58 (d, J=1.1 Hz, 1H), 7.57-7.55 (m, 1H), 7.40 (t, J=8.2 Hz, 1H), 6.92 (d, J=7.3 Hz, 1H).

### Example 23: N-(2-fluorophenyl)-4,6-bis(trifluoromethyl)-1H-benzo[d]imidazol-2-amine

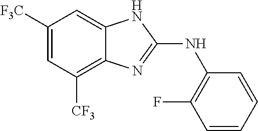 [SMILES: Fc1ccccc1Nc1nc2c(C(F)(F)F)cc(C(F)(F)F)cc2[nH]1]

Following a procedure analogous to the one provided for compound of example 9 but 2-fluoroaniline and intermediate-1 was used in this case to obtain the title compound as off-white solid.

LCMS (ESI): m/z=364.1 (M+H)⁺

¹H NMR (500 MHz, MeOD-d4): δ 7.94 (s, 2H), 7.72-7.66 (m, 1H), 7.60-7.53 (m, 1H), 7.47-7.39 (m, 2H).

### Example 24: N-(3-fluorophenyl)-4,6-bis(trifluoromethyl)-1H-benzo[d]imidazol-2-amine

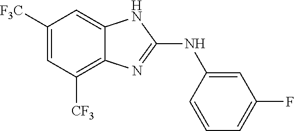 [SMILES: Fc1cccc(Nc2nc3c(C(F)(F)F)cc(C(F)(F)F)cc3[nH]2)c1]

Following a procedure analogous to the one provided for compound of example 9 but 3-fluoroaniline and intermediate-1 was used in this case to obtain the title compound as off-white solid.

LCMS (ESI): m/z=364.0 (M+H)⁺

¹H NMR (500 MHz, MeOD-d4): δ 7.84-7.80 (m, 2H), 7.49 (m, 1H), 7.31-7.26 (m, 2H), 7.13-7.08 (m, 1H).

### Example 25: N-(4-fluorophenyl)-4,6-bis(trifluoromethyl)-1H-benzo[d]imidazol-2-amine

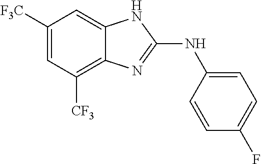 [SMILES: Fc1ccc(Nc2nc3c(C(F)(F)F)cc(C(F)(F)F)cc3[nH]2)cc1]

Following a procedure analogous to the one provided for compound of example 9 but 4-fluoroaniline and intermediate-1 was used in this case to obtain the title compound as off-white solid.

LCMS (ESI): m/z=364.0 (M+H)⁺

¹H NMR (500 MHz, MeOD-d4): δ 7.90 (d, J=0.4 Hz, 1H), 7.89 (s, 1H), 7.61-7.56 (m, 2H), 7.36-7.31 (m, 2H).

### Example 26: N-(3,5-difluorophenyl)-4,6-bis(trifluoromethyl)-1H-benzo[d]imidazol-2-amine

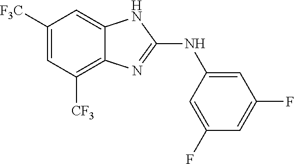 [SMILES: Fc1cc(F)cc(Nc2nc3c(C(F)(F)F)cc(C(F)(F)F)cc3[nH]2)c1]

Following a procedure analogous to the one provided for compound of example 9 but 3,5-difluoroaniline and intermediate-1 was used in this case to obtain the title compound as off-white solid.

LCMS (ESI): m/z=382.0 (M+H)⁺

¹H NMR (500 MHz, MeOD-d4): δ 7.97 (s, 1H), 7.91 (s, 1H), 7.27 (m, 2H), 7.03 (m, 1H).

### Example 27: 4,6-bis(trifluoromethyl)-N-(4-(trifluoromethyl)phenyl)-1H-benzo[d]imidazol-2-amine

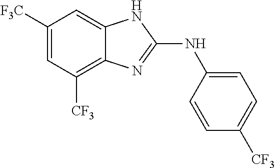 [SMILES: FC(F)(F)c1ccc(Nc2nc3c(C(F)(F)F)cc(C(F)(F)F)cc3[nH]2)cc1]

Following a procedure analogous to the one provided for compound of example 9 but 4-aminobenzotrifluoride and intermediate-1 was used in this case to obtain the title compound as off-white solid.

LCMS (ESI): m/z=414.0 (M+H)⁺

¹H NMR (500 MHz, MeOD-d4): δ 7.95 (s, 1H), 7.90 (s, 1H), 7.86 (d, J=8.5 Hz, 2H), 7.77 (d, J=8.4 Hz, 2H).

### Example 28: 4,6-bis(trifluoromethyl)-N-(2-(trifluoromethyl)phenyl)-1H-benzo[d]imidazol-2-amine

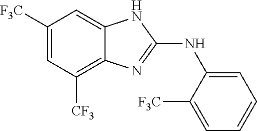 [SMILES: FC(F)(F)c1cc(C(F)(F)F)c2nc(Nc3ccccc3C(F)(F)F)[nH]c2c1]

Following a procedure analogous to the one provided for compound of example 9 but 2-aminobenzotrifluoride and intermediate-1 was used in this case to obtain the title compound as off-white solid.

LCMS (ESI): m/z=414.0 (M+H)⁺

¹H NMR (500 MHz, MeOD-d4): δ 7.85 (d, J=7.9 Hz, 1H), 7.79 (m, 4H), 7.62 (t, J=7.3 Hz, 1H).

### Example 29: N-(3,5-bis(trifluoromethyl)phenyl)-6-(trifluoromethoxy)-1H-benzo[d]imidazol-2-amine

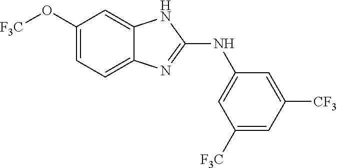 [SMILES: FC(F)(F)Oc1ccc2nc(Nc3cc(C(F)(F)F)cc(C(F)(F)F)c3)[nH]c2c1]

Following a procedure analogous to the one provided for compound of example 9 but 3,5-bis(trifluoromethyl)aniline and intermediate-3 was used in this case to obtain the title compound as off-white solid.

LCMS (ESI): m/z=430.0 (M+H)⁺

¹H NMR (500 MHz, DMSO-d6): δ 11.42 (bs, 1H), 8.38 (s, 2H), 7.79 (s, 1H), 7.52 (d, J=8.6 Hz, 1H), 7.45 (d, J=1.2 Hz, 1H), 7.18 (d, J=8.6 Hz, 1H).

### Example 30: 6-(trifluoromethoxy)-N-(3-(trifluoromethyl)phenyl)-1H-benzo[d]imidazol-2-amine

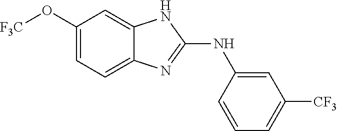 [SMILES: FC(F)(F)Oc1ccc2nc(Nc3cccc(C(F)(F)F)c3)[nH]c2c1]

Following a procedure analogous to the one provided for compound of example 9 but 3-aminobenzotrifluoride and intermediate-3 was used in this case to obtain the title compound as off-white solid.

LCMS (ESI): m/z=362.0 (M+H)⁺

¹H NMR (500 MHz, DMSO-d6): δ 11.86 (bs, 1H), 7.94 (s, 1H), 7.86 (d, J=8.0 Hz, 1H), 7.71 (t, J=7.9 Hz, 1H), 7.61 (d, J=7.8 Hz, 1H), 7.54 (d, J=8.7 Hz, 1H), 7.45 (d, J=1.2 Hz, 1H), 7.25 (dd, J=8.7, 1.4 Hz, 1H).

### Example 31: N-(3,5-bis(trifluoromethyl)phenyl)-6-(trifluoromethyl)-1H-benzo[d]imidazol-2-amine

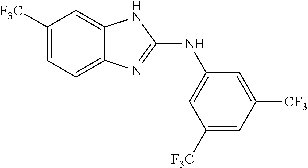 [SMILES: FC(F)(F)c1cc(Nc2nc3ccc(C(F)(F)F)cc3[nH]2)cc(C(F)(F)F)c1]

Following a procedure analogous to the one provided for compound of example 9 but 3,5-bis(trifluoromethyl)aniline and intermediate-4 was used in this case to obtain the title compound as off-white solid.

LCMS (ESI): m/z=414.0 (M+H)⁺

¹H NMR (500 MHz, MeOD-d4): δ 8.18 (s, 2H), 7.57 (s, 1H), 7.41 (s, 1H), 7.39 (dd, J=8.4, 1.3 Hz, 1H), 7.28 (d, J=8.2 Hz, 1H).

### Example 32: 6-(trifluoromethyl)-N-(3-(trifluoromethyl)phenyl)-1H-benzo[d]imidazol-2-amine

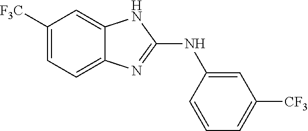 [SMILES: FC(F)(F)c1cccc(Nc2nc3ccc(C(F)(F)F)cc3[nH]2)c1]

Following a procedure analogous to the one provided for compound of example 9 but 3-aminobenzotrifluoride and intermediate-4 was used in this case to obtain the title compound as off-white solid.

LCMS (ESI): m/z=346.0 (M+H)⁺

¹H NMR (500 MHz, DMSO-d6): δ 11.49 (bs, 1H), 10.11 (bs, 1H), 8.29 (s, 1H), 8.02 (d, J=8.1 Hz, 1H), 7.67 (d, 1H), 7.57 (t, J=8.0 Hz, 1H), 7.52 (s, 1H), 7.36 (dd, J=8.3, 1.2 Hz, 1H), 7.30 (d, J=7.7 Hz, 1H).

### Example 33: 6-(trifluoromethyl)-N-(4-(trifluoromethyl)phenyl)-1H-benzo[d]imidazol-2-amine

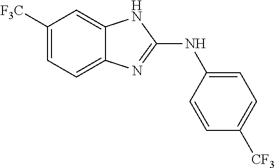 [SMILES: FC(F)(F)c1ccc(Nc2nc3ccc(C(F)(F)F)cc3[nH]2)cc1]

Following a procedure analogous to the one provided for compound of example 9 but 4-aminobenzotrifluoride and intermediate-4 was used in this case to obtain the title compound as off-white solid.

LCMS (ESI): m/z=346.0 (M+H)⁺

¹H NMR (500 MHz, DMSO-d6): δ 11.48 (bs, 1H), 10.20 (bs, 1H), 7.99 (d, J=8.5 Hz, 2H), 7.70 (d, J=8.7 Hz, 3H), 7.53 (d, J=8.2 Hz, 1H), 7.37 (dd, J=8.3, 1.3 Hz, 1H).

### Example 34: N-(3,5-bis(trifluoromethyl)phenyl)-4-(trifluoromethyl)-1H-benzo[d]imidazol-2-amine

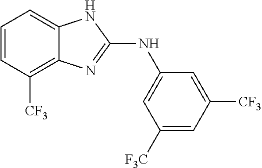 [SMILES: FC(F)(F)c1cc(Nc2nc3c(C(F)(F)F)cccc3[nH]2)cc(C(F)(F)F)c1]

Following a procedure analogous to the one provided for compound of example 9 but 3,5-bis(trifluoromethyl)aniline and intermediate-5 was used in this case to obtain the title compound as off-white solid.

LCMS (ESI): m/z=414.0 (M+H)⁺

¹H NMR (500 MHz, DMSO-d6): δ 10.79 (bs, 1H), 8.61 (s, 2H), 7.72-7.59 (m, 2H), 7.40 (d, J=7.7 Hz, 1H), 7.22 (t, J=7.8 Hz, 1H).

### Example 35: 4-(trifluoromethyl)-N-(3-(trifluoromethyl)phenyl)-1H-benzo[d]imidazol-2-amine

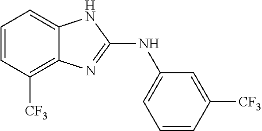 [SMILES: FC(F)(F)c1cccc(Nc2nc3c(C(F)(F)F)cccc3[nH]2)c1]

Following a procedure analogous to the one provided for compound of example 9 but 3-aminobenzotrifluoride and intermediate-5 was used in this case to obtain the title compound as off-white solid.

LCMS (ESI): m/z=346.0 (M+H)⁺

¹H NMR (500 MHz, DMSO-d6): δ 11.59 (bs, 1H), 10.16 (bs, 1H), 8.49 (s, 1H), 7.95 (d, J=8.1 Hz, 1H), 7.57 (dd, J=15.5, 7.8 Hz, 2H), 7.32 (dd, J=31.8, 7.7 Hz, 2H), 7.15 (t, J=7.8 Hz, 1H).

### Example 36: 4-(trifluoromethyl)-N-(4-(trifluoromethyl)phenyl)-1H-benzo[d]imidazol-2-amine

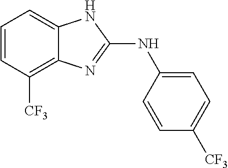 [SMILES: FC(F)(F)c1ccc(Nc2nc3c(C(F)(F)F)cccc3[nH]2)cc1]

Following a procedure analogous to the one provided for compound of example 9 but 4-aminobenzotrifluoride and intermediate-5 was used in this case to obtain the title compound as off-white solid.

LCMS (ESI): m/z=346.1 (M+H)⁺

¹H NMR (500 MHz, DMSO-d6): δ 11.59 (bs, 1H), 10.16 (bs, 1H), 8.49 (s, 1H), 7.95 (d, J=8.1 Hz, 1H), 7.61-7.52 (m, 2H), 7.35 (d, J=7.7 Hz, 1H), 7.28 (d, J=7.7 Hz, 1H), 7.15 (t, J=7.8 Hz, 1H).

### Example 37: N-(3,5-bis(trifluoromethyl)phenyl)-6-methoxy-1H-benzo[d]imidazol-2-amine

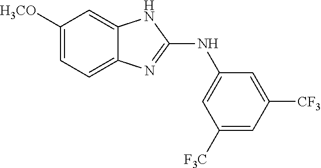 [SMILES: COc1ccc2nc(Nc3cc(C(F)(F)F)cc(C(F)(F)F)c3)[nH]c2c1]

Following a procedure analogous to the one provided for compound of example 9 but 3,5-bis(trifluoromethyl)aniline and intermediate-6 was used in this case to obtain the title compound as off-white solid.

LCMS (ESI): m/z=376.0 (M+H)⁺

¹H NMR (500 MHz, DMSO-d6): δ 11.23 (bs, 1H), 10.19 (bs, 1H), 8.50 (d, J=4.8 Hz, 2H), 7.53 (s, 1H), 7.28 (m, 1H), 6.98 (m, 1H), 6.69 (s, 1H), 3.77 (s, 3H).

### Example 38: 5,6-difluoro-1-methyl-N-phenyl-1H-benzo[d]imidazol-2-amine

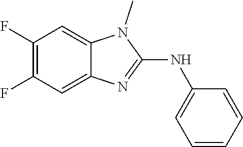 [SMILES: Cn1c(Nc2ccccc2)nc2cc(F)c(F)cc21]

Following a procedure analogous to the one provided for compound of example 9 but aniline and intermediate-7 was used in this case to obtain the title compound as off-white solid.

LCMS (ESI): m/z=260.1 (M+H)⁺

¹H NMR (500 MHz, MeOD-d4): δ 7.51 (dd, J=8.6, 1.0 Hz, 2H), 7.34-7.29 (m, 2H), 7.22-7.15 (m, 2H), 7.03 (m, 1H), 3.64 (s, 3H).

### Example 39: N-(3,5-bis(trifluoromethyl)phenyl)-5,6-difluoro-1-methyl-1H-benzo[d]imidazol-2-amine

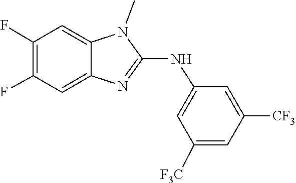 [SMILES: Cn1c(Nc2cc(C(F)(F)F)cc(C(F)(F)F)c2)nc2cc(F)c(F)cc21]

Following a procedure analogous to the one provided for compound of example 9 but 3,5-bis(trifluoromethyl)aniline and intermediate-7 was used in this case to obtain the title compound as off-white solid.

LCMS (ESI): m/z=396.0 (M+H)⁺

¹H NMR (500 MHz, DMSO-d6): δ 12.19 (bs, 1H), 8.06 (s, 1H), 7.86 (d, J=7.7 Hz, 1H), 7.74 (t, J=7.9 Hz, 1H), 7.67 (s, 1H), 7.59 (s, 1H), 3.64 (s, 3H).

### Example 40: N-methyl-4,6-bis(trifluoromethyl)-N-(3-(trifluoromethyl)phenyl)-1H-benzo[d]imidazol-2-amine

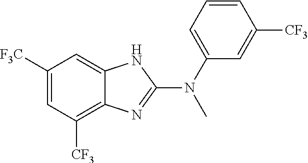 [SMILES: CN(c1cccc(C(F)(F)F)c1)c1nc2c(C(F)(F)F)cc(C(F)(F)F)cc2[nH]1]

Following a procedure analogous to the one provided for compound of example 9 but N-methyl-3-aminobenzotrifluoride and intermediate-1 was used in this case to obtain the title compound as off-white solid.

LCMS (ESI): m/z=428.0 (M+H)⁺

¹H NMR (500 MHz, DMSO-d6): δ 12.19 (bs, 1H), 8.06 (s, 1H), 7.86 (d, J=7.7 Hz, 1H), 7.74 (t, J=7.9 Hz, 1H), 7.67 (s, 2H), 7.59 (s, 1H), 3.64 (s, 3H).

### Example 41: N-(3,5-bis(trifluoromethyl)phenyl)-N-methyl-4,6-bis(trifluoromethyl)-1H-benzo[d]imidazol-2-amine

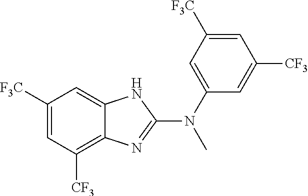 [SMILES: CN(c1cc(C(F)(F)F)cc(C(F)(F)F)c1)c1nc2c(C(F)(F)F)cc(C(F)(F)F)cc2[nH]1]

Following a procedure analogous to the one provided for compound of example 9 but N-methyl-3,5-bis(trifluoromethyl)aniline and intermediate-1 was used in this case to obtain the title compound as off-white solid.

LCMS (ESI): m/z=496.0 (M+H)⁺

¹H NMR (500 MHz, DMSO-d6): δ 12.64 (bs, 1H), 9.06 (s, 1H), 8.41 (s, 1H), 7.93 (s, 1H), 7.78 (s, 1H), 7.64 (s, 1H), 3.71 (s, 3H).

### Example 42: N-methyl-7-(trifluoromethyl)-N-(3-(trifluoromethyl)phenyl)-1H-benzo[d]imidazol-2-amine

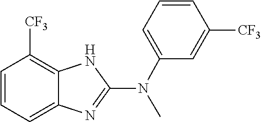 [SMILES: CN(c1cccc(C(F)(F)F)c1)c1nc2cccc(C(F)(F)F)c2[nH]1]

Following a procedure analogous to the one provided for compound of example 9 but N-methyl-3-aminobenzotrifluoride and intermediate-5 was used in this case to obtain the title compound as off-white solid.

LCMS (ESI): m/z=360.1 (M+H)⁺

¹H NMR (500 MHz, DMSO-d6): δ 11.91 (bs, 1H), 8.03 (s, 1H), 7.78 (d, J=7.6 Hz, 1H), 7.67 (t, J=7.9 Hz, 1H), 7.55 (d, J=7.6 Hz, 1H), 7.47 (d, J=7.1 Hz, 1H), 7.32 (d, J=7.7 Hz, 1H), 7.10 (t, J=7.4 Hz, 1H), 3.59 (s, 3H).

### Example 43: N-methyl-7-(trifluoromethyl)-N-(4-(trifluoromethyl)phenyl)-1H-benzo[d]imidazol-2-amine

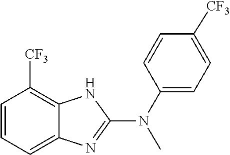 [SMILES: CN(c1ccc(C(F)(F)F)cc1)c1nc2cccc(C(F)(F)F)c2[nH]1]

Following a procedure analogous to the one provided for compound of example 9 but N-methyl-4-aminobenzotrifluoride and intermediate-5 was used in this case to obtain the title compound as white solid.

LCMS (ESI): m/z=360.1 (M+H)⁺

¹H NMR (500 MHz, DMSO-d6): δ 12.04 (bs, 1H), 7.77 (d, J=8.7 Hz, 2H), 7.70 (d, J=8.5 Hz, 2H), 7.50 (d, J=7.8 Hz, 1H), 7.34 (d, J=7.7 Hz, 1H), 7.13 (t, J=7.8 Hz, 1H), 3.61 (s, 3H).

### Example 44: N-(3,5-bis(trifluoromethyl)phenyl)-N-methyl-7-(trifluoromethyl)-1H-benzo[d]imidazol-2-amine

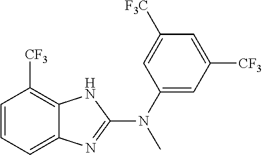 [SMILES: CN(c1cc(C(F)(F)F)cc(C(F)(F)F)c1)c1nc2cccc(C(F)(F)F)c2[nH]1]

Following a procedure analogous to the one provided for compound of example 9 but N-methyl-3,5-bis(trifluoromethyl)aniline and intermediate-5 was used in this case to obtain the title compound as off-white solid.

LCMS (ESI): m/z=428.0 (M+H)⁺

¹H NMR (500 MHz, DMSO-d6): δ 12.28 (bs, 1H), 8.38 (s, 2H), 7.80 (s, 1H), 7.57 (d, J=7.8 Hz, 1H), 7.37 (d, J=7.7 Hz, 1H), 7.17 (t, J=7.8 Hz, 1H), 3.67 (s, 3H).

### Antiviral Activity

Antiviral activity of the compounds was tested using cell-based anti-viral assay using Vero E6 cell line. The reduction in viral RNA in the presence of compounds was determined by qRT-PCR. Briefly, Vero E6 cells were seeded into a 48-well plate 1 day prior to the experiment. (For cell plating, cell count was maintained at 2.5×10⁵ cells/mL, confluency was maintained at more than 90%, and 5×10⁴ cells/well). Cells were pre-treated with the test compounds at the specified concentrations for 15 mins. After pre-treatment, the cells were infected with 50 μL of 1:1000 virus stock solution and incubated for 1 hour at 37° C. and 5% CO₂. Post infection, the cells were washed by using 1×PBS (100 μL), followed by addition of 200 μL of the fresh Advanced DMEM media (supplemented with 5% FBS, 1× Glutamax and 1× Antibiotic-Penicillin/Streptomycin) containing desired concentrations of the test compounds. VC104 (Niclosamide) and RMD (Remdesivir) were used as positive controls for the assay while CC (only cells control) were used as the Negative control. The cells were incubated for 48 h at 37° C. and 5% CO₂. After incubation, vRNA was extracted from the culture supernatant using GSure Viral RNA isolation kit (Fast-GCC Biotech), as per the manufacturer's recommendation and quantified by qRT-PCR assay using the SARS-CoV-2-specific primers (Diagsure RT PCR kit, GCC Biotech). The increase in Ct value (reduction in viral RNA) in the presence of drug was calculated with respect to the control where no drug was added. The difference in Ct value was used to calculate percent inhibition of SARS-CoV-2 by the compounds. All the cell-based antiviral assays were performed at biosafety level-3 (BSL-3) at CSIR-IMTECH.

| | ΔCt | % SARS-CoV-2 | ΔCt | % SARS-CoV-2 |
|---|---|---|---|---|
| Example | (5 μM) | Inactivation (5 μM) | (1 μM) | Inactivation (1 μM) |
| 1 | 12 | >99.9 | 5.7 | >95 |
| 2 | 6 | >95 | 4.4 | >90 |
| 3 | 3.3 | 90 | 1.8 | N/S |
| 4 | 6 | >95 | 2.7 | N/S |
| 5 | 1.2 | N/S | 1 | N/S |
| 6 | 1.6 | N/S | 2 | N/S |
| 7 | −0.32 | N/S | 0.9 | N/S |
| 8 | 4.2 | >90 | 3.1 | 90 |
| 9 | 1 | N/S | −1.1 | N/S |
| 10 | 2.4 | N/S | 2.4 | N/S |
| 11 | 10.3 | 99.9 | 5.8 | >95 |
| 12 | 3.5 | 90 | 2.4 | N/S |
| 13 | 1 | N/S | 1.3 | N/S |
| 14 | 16 | 99.99 | 7.4 | 99 |
| 15 | 4.2 | >90 | 3.1 | 90 |
| 16 | 1 | N/S | 0.8 | N/S |
| 17 | 1.5 | N/S | 3 | 90 |
| 18 | 1.4 | N/S | 1.4 | N/S |
| 19 | 1.9 | N/S | 1.9 | N/S |
| 20 | 3.7 | >90 | 1.7 | N/S |
| 21 | 9.3 | >99 | 3.6 | >90 |
| 22 | 7.4 | 99 | 3.3 | 90 |
| 23 | 5.2 | >95 | −0.3 | N/S |
| 24 | 4.3 | >90 | 0.7 | N/S |
| 25 | 3.6 | 90 | 2 | N/S |
| 26 | 9.4 | >99 | 3.4 | 90 |
| 27 | 8 | >99 | 1.2 | N/S |
| 28 | 2 | N/S | −2 | N/S |
| 29 | 11.5 | 99.9 | 4.7 | >95 |
| 30 | 11.1 | 99.9 | 1.1 | N/S |
| 31 | 9 | >99 | 5.3 | >95 |
| 32 | 9.3 | >99 | 0.2 | N/S |
| 33 | 14.3 | 99.99 | 1.4 | N/S |
| 34 | 15.1 | 99.99 | 13.1 | >99.9 |
| 35 | 7.5 | 99 | 1.7 | N/S |
| 36 | 12 | >99.9 | 12 | >99.9 |
| 37 | 4.3 | >90 | 5 | >95 |
| 38 | −0.1 | N/S | 0.4 | N/S |
| 39 | 1 | N/S | 0.5 | N/S |
| 40 | 12.2 | >99.9 | 5 | >95 |
| 41 | 13.4 | >99.9 | 3.8 | >90 |
| 42 | 2.1 | N/S | 1 | N/S |
| 43 | 3.6 | >90 | 1.8 | N/S |
| 44 | 5 | >95 | 1.4 | N/S |

*N/S- non significant (ΔCt value <3 was considered non-significant due to inherent variations in the RT-PCR based experimental data)

None of the above compounds showed any apparent cytotoxicity in the cell-based anti-viral assays tested at 1 and 5 μM concentrations.

Thus, the above in-vitro assay method showed that the compounds of the invention were found to have antiviral activity against coronavirus (SARS-CoV-2), thereby showing utility for treating diseases, and disorders associated with viral diseases particularly in the case of coronavirus.

The compound testing results have demonstrated that the substituted benzimidazole compounds of formula (I) are capable of treating/reducing viral infections.

All patents, patent applications and publications cited in this application are hereby incorporated by reference in their entirety for all purposes to the same extent as if each individual patent, patent application or publication were so individually denoted.

Although certain embodiments and examples have been described in detail above, those having ordinary skill in the art will clearly understand that many modifications are possible in the embodiments and examples without departing from the teachings thereof. All such modifications are intended to be encompassed within the below claims of the invention.

### Pharmaceutical Formulations

Effective amounts of the active compounds were used with suitable carriers or excipients to prepare pharmaceutical formulations. The carriers or excipients used were suitably selected from water, salt solutions, alcohols, polyethylene glycols, polyhydroxyethoxylated castor oil, peanut oil, olive oil, gelatin, lactose, terra alba, sucrose, dextrin, magnesium carbonate, sugar, cyclodextrin, amylose, magnesium stearate, talc, gelatin, agar, pectin, acacia, stearic acid or lower alkyl ethers of cellulose, salicylic acid, fatty acids, fatty acid amines, fatty acid monoglycerides and diglycerides, pentaerythritol fatty acid esters, polyoxyethylene, hydroxymethylcellulose and polyvinylpyrrolidone or combinations thereof. The compound of general Formula (I) can also be used with one or more antiviral compounds. Hence formulations of the effective amounts of the active compounds of general Formula (I) with one or more antiviral compounds are prepared.

1-10. (canceled)

11. A compound of general formula (I): 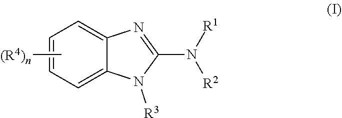
a stereoisomer, or a pharmaceutically acceptable salt thereof, where:
R¹ and R² are independently selected from hydrogen, substituted alkyl, unsubstituted alkyl, substituted cycloalkyl, unsubstituted cycloalkyl, m-substituted aryl, p-substituted aryl, unsubstituted aryl, substituted heteroaryl, unsubstituted heteroaryl, a substituted heterocyclic ring, or an unsubstituted heterocyclic ring, wherein substituents of substituted alkyl, cycloalkyl, aryl, heteroaryl, or heterocyclic rings are selected from fluorine, bromine, iodine, alkyl, alkoxy, oxo, nitro, trifluoromethyl, trifluoromethoxy, aryl, or heteroaryl;
R¹ and R², together with the nitrogen to which they are attached, optionally are joined may form a substituted or unsubstituted 3 to 7 membered saturated or unsaturated heterocyclic ring, wherein substituents of substituted heterocyclic rings are selected from halogen, alkyl, alkoxy, oxo, nitro, trifluoromethyl, trifluoromethoxy, aryl, or heteroaryl;
R¹ and R² are not both hydrogen;
R³ is selected from the group consisting of hydrogen, substituted alkyl, unsubstituted alkyl, substituted cycloalkyl, unsubstituted cycloalkyl, alkoxy, carboxy, carboxy ester, substituted aryl, unsubstituted aryl, substituted heteroaryl, and unsubstituted heteroaryl;
each R⁴ is independently selected from fluorine, bromine, iodine, nitro, hydroxyl, trifluoromethyl, trifluoromethoxy, substituted alkyl, substituted haloalkyl, substituted cycloalkyl, unsubstituted cycloalkyl, —NR^{a}R^{b}, —NR^{a}C(O)R^{b}, —NR^{a}S(O)₀₋₂R^{b}, —S(O)₀₋₂R^{a}, and —S(O)₀₋₂NR^{a}R^{b};
each R^{a} and each R^{b} is independently selected from hydrogen, substituted alkyl, unsubstituted alkyl, substituted cycloalkyl, unsubstituted cycloalkyl, substituted cycloalkylalkyl, unsubstituted cycloalkylalkyl, substituted aryl, unsubstituted aryl, substituted arylalkyl, unsubstituted arylalkyl, substituted heteroaryl, unsubstituted heteroaryl, substituted heteroarylalkyl, unsubstituted heteroarylalkyl, substituted heterocyclyl, unsubstituted heterocyclyl, substituted heterocyclylalkyl, or unsubstituted heterocyclylalkyl;
R^{a} and R^{b}, together with the nitrogen atom to which they are attached, optionally are joined to form a substituted or unsubstituted, saturated or unsaturated 3- to 10-membered cyclic ring; and
n is 1, 2, 3, or 4.

12. The compound of claim 11, wherein the compound of general formula (I) is selected from the group consisting of
N-(3,5-bis(trifluoromethyl)phenyl)-4,6-bis(trifluoromethyl)-1H-benzo[d]imidazol-2-amine;
N-phenyl-4,6-bis(trifluoromethyl)-1H-benzo[d]imidazol-2-amine;
N-(3-bromo-4-methoxyphenyl)-4,6-bis(trifluoromethyl)-1H-benzo[d]imidazol-2-amine;
N-(pyridin-3-yl)-4,6-bis(trifluoromethyl)-1H-benzo[d]imidazol-2-amine;
N-benzyl-4,6-bis(trifluoromethyl)-1H-benzo[d]imidazol-2-amine;
N-(tetrahydro-2H-pyran-4-yl)-4,6-bis(trifluoromethyl)-1H-benzo[d]imidazol-2-amine;
5,6-difluoro-N-phenyl-1H-benzo[d]imidazol-2-amine;
N-(3-bromo-4-methoxyphenyl)-5,6-difluoro-1H-benzo[d]imidazol-2-amine;
5,6-difluoro-N-(3-(trifluoromethyl)phenyl)-1H-benzo[d]imidazol-2-amine;
N-(3,5-bis(trifluoromethyl)phenyl)-5,6-difluoro-1H-benzo[d]imidazol-2-amine;
5,6-difluoro-N-(4-(trifluoromethyl)phenyl)-1H-benzo[d]imidazol-2-amine;
N-benzyl-5,6-difluoro-1H-benzo[d]imidazol-2-amine;
4,6-bis(trifluoromethyl)-N-(3-(trifluoromethyl)phenyl)-1H-benzo[d]imidazol-2-amine;
N-isopropyl-4,6-bis(trifluoromethyl)-1H-benzo[d]imidazol-2-amine;
2-(piperidin-1-yl)-4,6-bis(trifluoromethyl)-1H-benzo[d]imidazole;
(R)—N-(1-(naphthalen-1-yl)ethyl)-4,6-bis(trifluoromethyl)-1H-benzo[d]imidazol-2-amine;
3-((4,6-bis(trifluoromethyl)-1H-benzo[d]imidazol-2-yl)amino) tetrahydrothiophene 1,1-dioxide;
N-(1-methylcyclobutyl)-4,6-bis(trifluoromethyl)-1H-benzo[d]imidazol-2-amine;
N-(3-(trifluoromethoxy)phenyl)-4,6-bis(trifluoromethyl)-1H-benzo[d]imidazol-2-amine;
N-(3-fluorophenyl)-4,6-bis(trifluoromethyl)-1H-benzo[d]imidazol-2-amine;
N-(4-fluorophenyl)-4,6-bis(trifluoromethyl)-1H-benzo[d]imidazol-2-amine;
N-(3,5-difluorophenyl)-4,6-bis(trifluoromethyl)-1H-benzo[d]imidazol-2-amine;
4,6-bis(trifluoromethyl)-N-(4-(trifluoromethyl)phenyl)-1H-benzo[d]imidazol-2-amine;
N-(3,5-bis(trifluoromethyl)phenyl)-6-(trifluoromethoxy)-1H-benzo[d]imidazol-2-amine;
6-(trifluoromethoxy)-N-(3-(trifluoromethyl)phenyl)-1H-benzo[d]imidazol-2-amine;
N-(3,5-bis(trifluoromethyl)phenyl)-6-(trifluoromethyl)-1H-benzo[d]imidazol-2-amine;
6-(trifluoromethyl)-N-(3-(trifluoromethyl)phenyl)-1H-benzo[d]imidazol-2-amine;
6-(trifluoromethyl)-N-(4-(trifluoromethyl)phenyl)-1H-benzo[d]imidazol-2-amine;
N-(3,5-bis(trifluoromethyl)phenyl)-4-(trifluoromethyl)-1H-benzo[d]imidazol-2-amine;
4-(trifluoromethyl)-N-(3-(trifluoromethyl)phenyl)-1H-benzo[d]imidazol-2-amine;
4-(trifluoromethyl)-N-(4-(trifluoromethyl)phenyl)-1H-benzo[d]imidazol-2-amine;
N-(3,5-bis(trifluoromethyl)phenyl)-6-methoxy-1H-benzo[d]imidazol-2-amine;
5,6-difluoro-1-methyl-N-phenyl-1H-benzo[d]imidazol-2-amine;
N-(3,5-bis(trifluoromethyl)phenyl)-5,6-difluoro-1-methyl-1H-benzo[d]imidazol-2-amine;
N-methyl-4,6-bis(trifluoromethyl)-N-(3-(trifluoromethyl)phenyl)-1H-benzo[d]imidazol-2-amine;
N-(3,5-bis(trifluoromethyl)phenyl)-N-methyl-4,6-bis(trifluoromethyl)-1H-benzo[d]imidazol-2-amine;
N-methyl-7-(trifluoromethyl)-N-(3-(trifluoromethyl)phenyl)-1H-benzo[d]imidazol-2-amine;
N-methyl-7-(trifluoromethyl)-N-(4-(trifluoromethyl)phenyl)-1H-benzo[d]imidazol-2-amine; and
N-(3,5-bis(trifluoromethyl)phenyl)-N-methyl-7-(trifluoromethyl)-1H-benzo[d]imidazol-2-amine.

13. A process for preparing the compound of claim 11, the process comprising:
(a) acetylating a substituted aniline of formula (1): 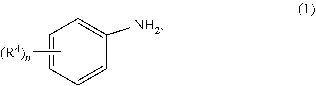
where R⁴ and n are as defined in the compound of formula (I), in the presence of an anhydride to produce a compound of formula (2): 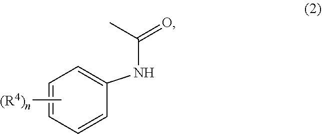
where R⁴ and n are as defined in the compound of formula (1);
(b) nitrating the compound of formula (2) to produce an acetamide compound of formula (3): 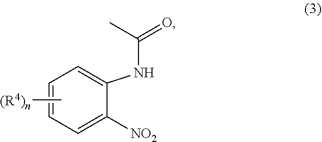
where R⁴ and n are as defined in the compound of formula (2);
(c) treating the acetamide compound with a base to produce a compound of formula (4): 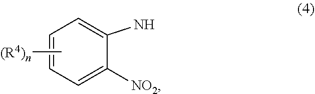
where R⁴ and n are as defined in the compound of formula (3);
(d) reducing a nitro group of the compound of formula (4) by a reducing agent to produce a diamine of formula (5): 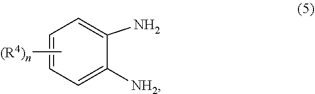
where R⁴ and n are as defined in the compound of formula (4);
(e) cyclizing the diamine with a coupling agent, optionally in the presence of a catalytic amount of a base, to produce a compound of formula (6): 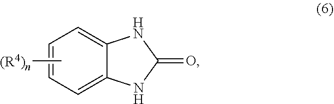
where R⁴ and n are as defined in the compound of formula (5);
(f) halogenating the compound of formula (6) to produce a compound of formula (7): 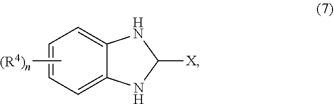
where R⁴ and n are as defined in the compound of formula (6);
(g) optionally performing a N-substitution of the compound of formula (7) with an alkylating/arylating agent in basic condition to obtain a compound of formula (9): 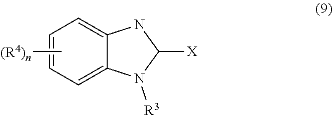
where R⁴ and n are as defined in the compound of formula (7); and
(h) performing under acidic conditions, with aniline or with an amine of formula NHR¹R² where R¹ and R² are as defined in the compound of formula (I), a nucleophilic substitution of the compound of formula (7), or a nucleophilic substitution of the compound of formula (9), to obtain the compound of Formula (I).

14. The process according to claim 13, wherein the anhydride of (a) is acetic anhydride.

15. The process according to claim 13, wherein the base of (c) is sodium hydroxide or potassium hydroxide.

16. The process according to claim 13, wherein the reducing agent of (d) is selected from H₂, Pd/C, iron in acetic acid, or SnCl₂.

17. The process according to claim 13, wherein the coupling agent of (e) is carbonyldiimidazole or triphosgene.

18. The process according to in claim 13, wherein halogenation in (f) is carried out with phosphoryl chloride or phosphoryl bromide.

19. A pharmaceutical composition for treating coronavirus infections caused by SARS-CoV, MERS-CoV, SARS-CoV-2, and/or other viral infections, the pharmaceutical composition comprising the compound of claim 11 and at least one pharmaceutically acceptable excipient.

20. A pharmaceutical composition comprising the compound of claim 11 and at least one antiviral compound.
